(19) United States
(12) Patent Application Publication
(10) Pub. No.: US 2017/0051324 A1
(43) Pub. Date: Feb. 23, 2017

(54) Method for Producing L-Amino Acids in Corynebacteria Using a Glycine Cleavage System

(71) Applicant: EVONIK DEGUSSA GMBH, Essen (DE)

(72) Inventors: Ines OCHROMBEL, Bielefeld (DE); Brigitte BATHE, Salzkotten (DE); Marleen HASSELMEYER, Paderborn (DE)

(73) Assignee: Evonik Degussa GmbH, Essen (DE)

(21) Appl. No.: 15/307,368

(22) PCT Filed: Apr. 16, 2015
(86) PCT No.: PCT/EP2015/058280

§ 371 Date: Oct. 28, 2016

(30) Foreign Application Priority Data
Apr. 30, 2014 (EP) 14166649.5

(51) Int. Cl. C12P 13/12; C12N 9/10; C12P 13/14; C12N 9/02; C12P 13/06; C12P 13/08; C12N 9/06; C12N 9/12

(52) U.S. Cl. CPC C12P 13/12; C12N 9/0014; C12N 9/1014; C12N 9/1029; C12N 9/13; C12N 9/1241; C12N 9/0051; C12Y 104/04002; C12Y 108/01004; C12Y 201/0201; C12Y 203/01204; C12Y 203/01181; C12Y 208/01008; C12Y 207/07063; C12P 13/06; C12P 13/08; C12P 13/14

(57) ABSTRACT

It has been found, surprisingly, that the Corynebacterium humireducens strain comprises a very effective glycine cleavage system.

The present invention relates to a method for producing L-amino acids in Corynebacteria in which a glycine cleavage system is used.

Methods for producing L-amino acids that use bacteria from the genus Corynebacterium are known to those skilled in the art.

Although numerous Corynebacterium species are known, bacteria of the Corynebacterium glutamicum species are normally used in these methods, since this species has been found to be particularly advantageous for producing L-amino acids.

It has also been found that the yield of L-amino acid may be further increased by using a glycine cleavage system (GCV), since the formation of the undesired L-glycine by-product can be largely prevented by the glycine cleavage system. The suppression of the formation of L-glycine or of the degradation of excess L-glycine is particularly of particular significance in the production of L-methionine, since L-glycine is produced here as equimolar by-product.

A glycine cleavage system is composed of two or more subunits, namely the subunits GcvP, GcvT and GcvH. It takes the form of a multi-enzyme complex, which catalyses the oxidative decarboxylation and deamination of glycine to carbon dioxide, ammonium ions and N⁵⁻¹⁰-methylenetetrahydrofolate.

The glycine dehydrogenase GcvP is a pyridoxal phosphate-containing decarboxylase which liberates carbon dioxide and leaves the aminomethyl compound bound to pyridoxal phosphate.

The H-protein GcvH is a lipoamide-containing aminomethyltransferase.

The enzyme GcvT catalyses the nucleophilic attack on the aminomethyllipoamide by tetrahydrofolate forming N⁵⁻¹⁰-methylenetetrahydrofolate and liberating ammonium ions, wherein fully reduced lipoamide remains bound to GcvH.

Glycine cleavage systems are not present in all Corynebacteria, but to date only very few Corynebacteria have been described. For instance, the production strain C. glutamicum particularly preferred for amino acid production, for example, has no inherent glycine cleavage system. In order to utilise the advantages of a glycine cleavage system for C. glutamicum, such a system had to be incorporated into C. glutamicum from another Corynebacterium and be expressed heterologously. The glycine cleavage system from C. jeikeium (WO 2008/101857) has been used to date for this purpose. A serious disadvantage in this case is that C. jeikeium is a pathogenic organism. A further disadvantage is that, for the purpose of degradation of the undesired glycine by-product, a heterologous construct had to be prepared.

It was a primary object of the present invention, therefore, to provide a novel glycine cleavage system, preferably a glycine cleavage system that does not originate from a pathogenic organism, wherein the glycine cleavage system is preferably suitable for incorporation into other Corynebacteria, in particular, C. glutamicum.

A further object of the present invention was to provide a Corynebacterium that is intrinsically capable of producing L-amino acids and furthermore has an inherent glycine cleavage system such that the preparation of a heterologous construct—as in the case of C. glutamicum—is not necessary.

It has now been found in accordance with the invention, surprisingly, that the species Corynebacterium humireducens, which is non-pathogenic, has an inherent glycine cleavage system which is structurally significantly different from the glycine cleavage systems described to date.

It has further been found, surprisingly, that the glycine cleavage system of Corynebacterium humireducens is very effective, such that glycine, which is an undesirable by-product in amino acid synthesis, only accumulates in the cell in relatively low amounts, if at all, in the wild-type strain. By amplification of a glycine cleavage system according to the invention, particularly by overexpression, the suppression of glycine by-product formation can be correspondingly further improved. In particular, the glycine by-product formation can also be effectively suppressed by overexpression of a glycine cleavage system according to the invention in other microorganisms, particularly in C. glutamicum.

It has furthermore been found, surprisingly, that C. humireducens is even intrinsically capable of producing L-amino acids, particularly L-alanine, L-valine and L-glutamic acid, via the inherent requirement thereof, such that C. humireducens is an L-amino acid-producing strain having a homologous glycine cleavage system which may be employed particularly as a starting point for the development of further production strains having a host glycine cleavage system.

The C. humireducens strain is described for the first time by Wu et al. (International Journal of Systematic and Evolutionary Microbiology (2011), 61, 882-887). Said strain was deposited in the DSMZ under the deposition number DSM 45392 and its 16S rRNA was deposited in the EMBL and has the accession number GQ421281. The parent strain is a halotolerant, alkaliphilic, humic acid-reducing bacterium.

Further information regarding C. humireducens are to be found in the following publications: Wu et al. (Microb. Biotechnol. (2013), 6(2), 141-149), Lin et al. (Bioresour. Technol. (2013), 136, 302-308).

A first object of the present invention therefore relates to the enzymes of a glycine cleavage system selected from the group consisting of:
a) an enzyme GcvP having a sequence identity of at least 80%, preferably at least 85 or 90%, particularly at least 92, 94, 96 or 98%, especially 100%, to the sequence according to SEQ ID NO: 40,
b) an enzyme GcvT having a sequence identity of at least 80%, preferably at least 85 or 90%, particularly at least 92, 94, 96 or 98%, especially 100%, to the sequence according to SEQ ID NO: 42,
c) an enzyme GcvH having a sequence identity of at least 80%, preferably at least 85 or 90%, particularly at least 92, 94, 96 or 98%, especially 100%, to the sequence according to SEQ ID NO: 38.

The present invention therefore further relates to a glycine cleavage system comprising the enzymes GcvP, GcvT and GcvH, characterized in that said system comprises at least one of the following polypeptides:
a) an enzyme GcvP having a sequence identity of at least 80%, preferably at least 85 or 90%, particularly at least 92, 94, 96 or 98%, especially 100%, to the sequence according to SEQ ID NO: 40,
b) an enzyme GcvT having a sequence identity of at least 80%, preferably at least 85 or 90%, particularly at least 92, 94, 96 or 98%, especially 100%, to the sequence according to SEQ ID NO: 42,
c) an enzyme GcvH having a sequence identity of at least 80%, preferably at least 85 or 90%, particularly at least 92, 94, 96 or 98%, especially 100%, to the sequence according to SEQ ID NO: 38.

In this case, a glycine cleavage system according to the invention preferably comprises at least two, preferably all three, of the polypeptides (a) to (c) mentioned above.

Particular preference is given here to a glycine cleavage system comprising the following three polypeptides:
a) an enzyme GcvP having a sequence identity of at least 95 or 98% to the sequence according to SEQ ID NO: 40,
b) an enzyme GcvT having a sequence identity of at least 95 or 98% to the sequence according to SEQ ID NO: 42,
c) an enzyme GcvH having a sequence identity of at least 95 or 98% to the sequence according to SEQ ID NO: 38.

In a broader sense, the enzymes LpdA, LplA, LipA, LipB and GcvH also belong to the glycine cleavage system.

The dihydrolipoamide dehydrogenase (LpdA) reoxidises the lipoamide bound to GcvH.

The lipoate-protein ligase A (LplA) catalyses the lipoylation of GcvH.

The lipoic acid synthase (LipA) catalyses the synthesis of lipoic acid.

The lipoyl-[acyl carrier protein]-protein N-lipoyltransferase (LipB) catalyses the transfer of lipoic acid to the GcvH.

GcvL is a dihydrolipoyl dehydrogenase.

In a preferred embodiment, a glycine cleavage system according to the invention therefore further comprises at least one further enzyme selected from the group consisting of:
a) an enzyme LipA, preferably an enzyme LipA having a sequence identity of at least 80%, preferably at least 85 or 90%, particularly at least 92, 94, 96 or 98%, especially 100%, to the sequence according to SEQ ID NO: 48,
b) an enzyme LipB, preferably an enzyme LipB having a sequence identity of at least 80%, preferably at least 85 or 90%, particularly at least 92, 94, 96 or 98%, especially 100%, to the sequence according to SEQ ID NO: 50,
c) an enzyme Lpd, preferably an enzyme Lpd having a sequence identity of at least 80%, preferably at least 85 or 90%, particularly at least 92, 94, 96 or 98%, especially 100%, to the sequence according to SEQ ID NO: 52,
d) an enzyme LplA, preferably an enzyme LplA having a sequence identity of at least 80%, preferably at least 85 or 90%, particularly at least 92, 94, 96 or 98%, especially 100%, to the sequence according to SEQ ID NO: 94,
e) an enzyme GcvL, preferably an enzyme GcvL having a sequence identity of at least 80%, preferably at least 85 or 90%, particularly at least 92, 94, 96 or 98%, especially 100%, to the sequence according to SEQ ID NO: 96.

In a particularly preferred embodiment in accordance with the invention, a glycine cleavage system according to the invention in this case comprises at least two, three or four, preferably all five, of the polypeptides mentioned above.

The present invention therefore further relates also to enzymes selected from the group consisting of:
a) an enzyme LipA, preferably an enzyme LipA having a sequence identity of at least 80%, preferably at least 85 or 90%, particularly at least 92, 94, 96 or 98%, especially 100%, to the sequence according to SEQ ID NO: 48,
b) an enzyme LipB, preferably an enzyme LipB having a sequence identity of at least 80%, preferably at least 85 or 90%, particularly at least 92, 94, 96 or 98%, especially 100%, to the sequence according to SEQ ID NO: 50,
c) an enzyme Lpd, preferably an enzyme Lpd having a sequence identity of at least 80%, preferably at least 85 or 90%, particularly at least 92, 94, 96 or 98%, especially 100%, to the sequence according to SEQ ID NO: 52,
d) an enzyme LplA, preferably an enzyme LplA having a sequence identity of at least 80%, preferably at least 85 or 90%, particularly at least 92, 94, 96 or 98%, especially 100%, to the sequence according to SEQ ID NO: 94,
e) an enzyme GcvL, preferably an enzyme GcvL having a sequence identity of at least 80%, preferably at least 85 or 90%, particularly at least 92, 94, 96 or 98%, especially 100%, to the sequence according to SEQ ID NO: 96.

As an alternative to or in addition to the use of the appropriate enzymes which ensure an adequate synthesis of lipoic acid and/or lipoamide, these compounds may also be added to the reaction medium, for example, in amounts of up to 15 mM.

The present invention further relates also to polynucleotides coding for enzymes according to the invention and/or a glycine cleavage system according to the invention. These polynucleotides are preferably selected from the group consisting of:
a) a polynucleotide (gcvP), which codes for the enzyme GcvP and has a sequence identity of at least 70 or 75%, preferably at least 80 or 85%, particularly at least 90 or 95%, especially at least 98 or 100%, to the sequence of position 301 to 3162 according to SEQ ID NO: 39 and/or hybridizes under stringent conditions with a polynucleotide of which the sequence is complementary to the sequence of position 301 to 3162 according to SEQ ID NO: 39;
b) a polynucleotide (gcvT), which codes for the enzyme GcvT and has a sequence identity of at least 70 or 75%, preferably at least 80 or 85%, particularly at least 90 or 95%, especially at least 98 or 100%, to the sequence of position 301 to 1404 according to SEQ ID NO: 41 and/or hybridizes under stringent conditions with a polynucleotide of which the sequence is complementary to the sequence of position 301 to 1404 according to SEQ ID NO: 41;
c) a polynucleotide (gcvH), which codes for the enzyme GcvH and has a sequence identity of at least 70 or 75%, preferably at least 80 or 85%, particularly at least 90 or 95%, especially at least 98 or 100%, to the sequence of position 301 to 699 according to SEQ ID NO: 37 and/or hybridizes under stringent conditions with a polynucleotide of which the sequence is complementary to the sequence of position 301 to 699 according to SEQ ID NO: 37;
d) a polynucleotide (lipA), which codes for an enzyme LipA and has a sequence identity of at least 70%, preferably at least 80 or 85%, particularly at least 90 or 95%, especially at least 98 or 100%, to the sequence of position 301 to 1344 according to SEQ ID NO: 47 and/or hybridizes under stringent conditions with a polynucleotide of which the sequence is complementary to the sequence of position 301 to 1344 according to SEQ ID NO: 47;
e) a polynucleotide (lipB), which codes for an enzyme LipB and has a sequence identity of at least 70%, preferably at least 80 or 85%, particularly at least 90 or 95%, especially at least 98 or 100%, to the sequence of position 301 to 1089 according to SEQ ID NO: 49 and/or hybridizes under stringent conditions with a polynucleotide of which the sequence is complementary to the sequence of position 301 to 1089 according to SEQ ID NO: 49;
f) a polynucleotide (lpd), which codes for an enzyme Lpd and has a sequence identity of at least 70%, preferably at least 80 or 85%, particularly at least 90 or 95%, especially at least 98 or 100%, to the sequence of position 301 to 1710 according to SEQ ID NO: 51 and/or hybridizes under stringent conditions with a polynucleotide of which the sequence is complementary to the sequence of position 301 to 1710 according to SEQ ID NO: 51;
g) a polynucleotide (lplA), which codes for an enzyme LplA and has a sequence identity of at least 70%, preferably at least 80 or 85%, particularly at least 90 or 95%, especially at least 98 or 100%, to the sequence of position 301 to 1089 according to SEQ ID NO: 93 and/or hybridizes under stringent conditions with a polynucleotide of which the sequence is complementary to the sequence of position 301 to 1089 according to SEQ ID NO: 93;
h) a polynucleotide (gcvL), which codes for an enzyme GcvL and has a sequence identity of at least 70%, preferably at least 80 or 85%, particularly at least 90 or 95%, especially at least 98 or 100%, to the sequence of position 301 to 1710 according to SEQ ID NO: 95 and/or hybridizes under stringent conditions with a polynucleotide of which the sequence is complementary to the sequence of position 301 to 1710 according to SEQ ID NO: 95.

In accordance with the invention, “stringent conditions” is understood to mean washing at a salt concentration of 1×SSC and 0.1% by weight SDS at a temperature of 80° C.

The present invention further relates likewise to polynucleotides which are complementary to the coding polynucleotides mentioned above.

The present invention further relates to appropriate vectors, particularly cloning and expression vectors, comprising at least one polynucleotide according to the invention. These vectors can be appropriately incorporated into microorganisms, particularly in coryneform bacteria, especially from the genus Corynbebacterium, or Enterobacteriaceae, especially from the genus Escherichia.

Vectors according to the invention may comprise one or more polynucleotides in accordance with the invention. A preferred vector in accordance with the invention comprises at least one polynucleotide coding for an enzyme according to the invention selected from GcvP, GcvT and GcvH. A particularly preferred vector comprises polynucleotides which code for all three enzymes GcvP, GcvT and GcvH in accordance with the invention.

Vectors according to the invention preferably have a suitable promoter and/or suitable promoters and optionally further regulatory elements which enable the expression of the inventive polynucleotides in the recombinant bacterium, preferably the recombinant Corynebacterium.

Furthermore, polynucleotides according to the invention may also, for the purpose of expression of the coded genes, be incorporated into the genome of microorganisms, particularly into the genome of coryneform bacteria, particularly those of the genus Corynebacterium, or into the genome of Enterobacteriaceae, especially of the genus Escherichia.

The present invention further relates also to corresponding recombinant microorganisms, preferably bacteria, particularly coryneform bacteria, especially those of the genus Corynebacterium, particularly preferably of the species C. humireducens or C. glutamicum, and also Enterobacteriaceae, especially those of the genus Escherichia, comprising at least one enzyme according to the invention and/or at least one polynucleotide according to the invention and/or at least one vector according to the invention and/or one glycine cleavage system according to the invention and/or a polynucleotide coding for a glycine cleavage system according to the invention.

A preferred object is, in this context, recombinant Corynebacteria, particularly of the species C. humireducens and the species C. glutamicum, comprising at least one enzyme according to the invention, preferably all enzymes according to the invention, selected from GcvP, GcvT and GcvH, and/or polynucleotides coding for said enzymes and/or at least one vector comprising said polynucleotides.

The present invention particularly relates also to, in particular, recombinant microorganisms, preferably bacteria, particularly coryneform bacteria, especially those of the genus Corynebacterium with the exception of the species C. humireducens, particularly those of the species C. glutamicum, comprising at least one enzyme according to the invention and/or at least one polynucleotide according to the invention and/or at least one vector according to the invention and/or one glycine cleavage system according to the invention and/or a polynucleotide coding for a glycine cleavage system according to the invention.

A preferred object is, in this context, recombinant Corynebacteria, with the exception of the species C. humireducens, particularly of the species C. glutamicum, comprising at least one enzyme according to the invention, preferably all enzymes according to the invention, selected from GcvP, GcvT and GcvH, and/or polynucleotides coding for said enzymes and/or at least one vector comprising said polynucleotides.

In accordance with the invention, “recombinant microorganism” or “recombinant bacterium” is understood to mean a microorganism or bacterium that has been subjected to at least one genetic engineering measure. The genetic engineering measure may be, in this context, in particular a targeted or random mutation, the incorporation of a foreign gene and/or the overexpression or attenuation of a host gene or foreign gene. A recombinant microorganism according to the invention or a recombinant bacterium according to the invention is preferably characterized by the overexpression or attenuation of at least one gene. In a particularly preferred embodiment, a recombinant microorganism according to the invention or a recombinant bacterium according to the invention is characterized by the overexpression of at least one enzyme according to the invention and/or polynucleotide coding for said enzyme and/or the overexpression of a glycine cleavage system according to the invention or polynucleotide coding for said system.

“Relatively low amount”, with respect to glycine formation, is understood to mean an amount of at most 0.3 g/l, preferably at most 0.2 or 0.1 g/l, particularly preferably at most 0.05 or at most 0.03 g/l, based in each case on the accumulated glycine content in the cell and/or in the fermentation medium after completion of the fermentation.

Within the genus Corynebacterium, preference is given to strains according to the invention based on the following species: Corynebacterium efficiens, such as type strain DSM44549, Corynebacterium glutamicum, such as type strain ATCC13032 or the strain R, Corynebacterium ammoniagenes, such as type strain ATCC6871, Corynebacterium humireducens, such as the strain DSM 45392, and Corynebacterium pekinese, such as the strain CGMCC No. 5361.

Particular preference is given to the species Corynebacterium glutamicum and Corynebacterium humireducens. If, in the context of this application, the strain Corynebacterium humireducens is mentioned, said strain is preferably strain DSM 45392 or a strain derived therefrom.

Some representatives of the species Corynebacterium glutamicum are also known in the prior art under other names. These include for example: Corynebacterium acetoacidophilum ATCC13870, Corynebacterium lilium DSM20137, Corynebacterium melassecola ATCC17965, Brevibacterium flavum ATCC14067, Brevibacterium lactofermentum ATCC13869 and Brevibacterium divaricatum ATCC14020. The term “Micrococcus glutamicus” for Corynebacterium glutamicum has likewise been in use. Some representatives of the species Corynebacterium efficiens have also been referred to in the prior art as Corynebacterium thermoaminogenes, for example the strain FERM BP-1539.

Information on the taxonomic classification of strains of the group of the coryneform bacteria can be found, inter alia, in Seiler (Journal of General Microbiology 129, 1433-1477 (1983)), Kinoshita (1985, Glutamic Acid Bacteria, p 115-142. In: Demain and Solomon (ed), Biology of Industrial Microorganisms. The Benjamin/Cummins Publishing Co., London, UK), Kampfer and Kroppenstedt (Canadian Journal of Microbiology 42, 989-1005 (1996)), Liebl et al (International Journal of Systematic Bacteriology 41, 255-260 (1991)), Fudou et al (International Journal of Systematic and Evolutionary Microbiology 52, 1127-1131 (2002)) and in U.S. Pat. No. 5,250,434.

Strains with the designation “ATCC” may be obtained from the American Type Culture Collection (Manassas, Va., USA). Strains with the designation “DSM” may be obtained from the Deutschen Sammlung von Mikroorganismen und Zellkulturen (German Microorganism and Cell Culture Collection) (DSMZ, Braunschweig, Germany). Strains with the designation “NRRL” may be obtained from the Agricultural Research Service Patent Culture Collection (ARS, Peoria, Ill., US). Strains with the designation “FERM” may be obtained from the National Institute of Advanced Industrial Science and Technology (AIST Tsukuba Central 6, 1-1-1 Higashi, Tsukuba Ibaraki, Japan). Strains with the designation “CGMCC” may be obtained from the China General Microbiological Culture Collection Center (CGMCC, Beijing, China).

The present invention further relates also to a method for overproducing an L-amino acid, characterized in that at least one enzyme according to the invention and/or a polynucleotide coding for said enzyme and/or a glycine cleavage system according to the invention and/or polynucleotides coding for said system and/or a recombinant microorganism according to the invention, preferably a recombinant bacterium according to the invention, particularly a recombinant coryneform bacterium according to the invention, particularly preferably a recombinant Corynebacterium according to the invention, especially a Corynebacterium of the species C. humireducens or C. glutamicum, is used in said method. In a preferred embodiment according to the invention, the at least one polynucleotide according to the invention is used in this case in overexpressed form.

The present invention in this case preferably relates to a method for overproducing an L-amino acid, characterized in that a glycine cleavage system comprising the enzymes GcvP, GcvT and GcvH is used in said method, wherein the glycine cleavage system comprises at least one of the enzymes GcvP, GcvT and GcvH in accordance with the invention, and/or polynucleotides coding for the GcvP, GcvT and GcvH enzymes of a glycine cleavage system are used in said method, wherein said polynucleotides comprise at least one of the polynucleotides gcvP, gcvT and gcvH in accordance with the invention, and/or a recombinant Corynebacterium is used, preferably of the species C. humireducens or C. glutamicum, which comprises at least one enzyme according to the invention, preferably all three enzymes according to the invention, selected from GcvP, GcvT and GcvH and/or at least one polynucleotide according to the invention, preferably all three polynucleotides according to the invention, selected from gcvP, gcvT and gcvH.

The present invention in this case particularly preferably relates to a method for overproducing an L-amino acid, characterized in that a glycine cleavage system is used in said method, which comprises the enzymes GcvP, GcvT and GcvH in accordance with the invention and/or polynucleotides coding for said enzymes, and/or a recombinant Corynebacterium is used in said method, preferably of the species C. humireducens or C. glutamicum, which comprises such a glycine cleavage system and/or polynucleotides coding for said system.

“L-amino acid” in accordance with the invention is understood to mean, in particular, the proteinogenic L-amino acids.

The L-amino acid is in this case preferably selected from L-alanine, L-valine, L-amino acids of the glutamate family, particularly L-glutamate, L-glutamine, L-proline and L-arginine, and L-amino acids of the aspartate family, particularly L-aspartate, L-asparagine, L-methionine, L-lysine, L-isoleucine and L-threonine, particularly preferably selected from L-alanine, L-valine, L-glutamate, L-methionine, L-lysine and L-threonine. Particular preference is given to L-methionine.

The overproduction of the L-amino acids is preferably effected, in accordance with the invention, in C. humireducens or C. glutamicum.

Methods according to the invention are preferably characterized in that only low amounts of glycine occur as by-product. In the method according to the invention, glycine occurs preferably in an amount of less than 0.2 g/l, particularly in an amount of less than 0.1 g/l, particularly preferably in an amount of less than 0.05 g/l.

“Overproduce” or “overproduction” in relation to the L-amino acids is understood to mean, in accordance with the invention, that the microorganisms produce the L-amino acids according to their own requirement thereof, which either enrich in the cell or are secreted into the surrounding nutrient medium where they accumulate. In this case, the microorganisms preferably have the ability to enrich or accumulate in the cell or in the nutrient medium ≧ (at least) 0.25 g/l, ≧0.5 g/l, ≧1.0 g/l, ≧1.5 g/l, ≧2.0 g/l, ≧4 g/l or ≧10 g/l of the relevant L-amino acids in ≦ (at most) 120 hours, ≦96 hours, ≦48 hours, ≦36 hours, ≦24 hours or ≦12 hours.

Recombinant microorganisms according to the invention, in which polynucleotides according to the invention and/or vectors according to the invention have been incorporated, already have the capability, in a preferred embodiment, to overproduce an L-amino acid before the incorporation of the polynucleotides and/or vectors according to the invention. The starting strains are preferably strains which have been produced by mutagenesis and selection, by recombinant DNA techniques or by a combination of both methods.

It is obvious and requires no further explanation, that a recombinant microorganism in accordance with the invention can also be thus produced, in which a wild strain, in which a polynucleotide according to the invention and/or a vector according to the invention is present or has been incorporated and by subsequent suitable further genetic engineering measures, causes the L-amino acid to be produced or the L-amino acid production to be increased.

The present invention further relates also to other polynucleotides from C. humireducens and also the polypeptides encoded by said polynucleotides. By means of overexpression of the relevant polynucleotides or polypeptides, the amino acid production of certain L-amino acids can be positively influenced.

The present invention therefore likewise relates to:
a) a threonine dehydratase (llvA, EC 4.3.1.19) having a sequence identity of at least 90, 95 or 98%, preferably 100%, to the sequence according to SEQ ID NO: 106 and polynucleotides coding for the same,
b) the subunit of an acetolactate synthase (llvB) having a sequence identity of at least 90, 95 or 98%, preferably 100%, to the sequence according to SEQ ID NO: 98 and polynucleotides coding for the same,
c) an isomeroreductase (llvC, EC 1.1.1.86) having a sequence identity of at least 90, 95 or 98%, preferably 100%, to the sequence according to SEQ ID NO: 100 and polynucleotides coding for the same,
d) a dihydroxyacid dehydratase (llvD, EC 4.2.1.9) having a sequence identity of at least 90, 95 or 98%, preferably 100%, to the sequence according to SEQ ID NO: 102 and polynucleotides coding for the same,
e) a transaminase (llvE, EC 2.6.1.42) having a sequence identity of at least 90, 95 or 98%, preferably 100%, to the sequence according to SEQ ID NO: 104 and polynucleotides coding for the same,
f) an acetolactate synthase (llvH, EC 2.2.1.6) having a sequence identity of at least 90, 95 or 98%, preferably 100%, to the sequence according to SEQ ID NO: 122 and polynucleotides coding for the same,
g) a 3-methyl-2-oxobutanoate hydroxmethyltransferase (PanB, EC 2.1.2.11) having a sequence identity of at least 90, 95 or 98%, preferably 100%, to the sequence according to SEQ ID NO: 118 and polynucleotides coding for the same,
h) a pantothenate synthase (PanC, EC 6.3.2.1) having a sequence identity of at least 90, 95 or 98%, preferably 100%, to the sequence according to SEQ ID NO: 120 and polynucleotides coding for the same,
i) a glutamate dehydrogenase (Gdh) having a sequence identity of at least 90, 95 or 98%, preferably 100%, to the sequence according to SEQ ID NO: 124 and polynucleotides coding for the same,
j) a glutamine synthetase (glutamine synthetase 1) having a sequence identity of at least 90, 95 or 98%, preferably 100%, to the sequence according to SEQ ID NO: 126 and polynucleotides coding for the same,
k) a glutamine synthetase (glutamine synthetase 2) having a sequence identity of at least 90, 95 or 98%, preferably 100%, to the sequence according to SEQ ID NO: 128 and polynucleotides coding for the same,
l) a glutamate synthase having a sequence identity of at least 90, 95 or 98%, preferably 100%, to the sequence according to SEQ ID NO: 130 and polynucleotides coding for the same,
m) an isocitrate dehydrogenase having a sequence identity of at least 90, 95 or 98%, preferably 100%, to the sequence according to SEQ ID NO: 132 and polynucleotides coding for the same,
n) an aconitate hydrase having a sequence identity of at least 90, 95 or 98%, preferably 100%, to the sequence according to SEQ ID NO: 134 and polynucleotides coding for the same,
o) a citrate synthase having a sequence identity of at least 90, 95 or 98%, preferably 100%, to the sequence according to SEQ ID NO: 136 and polynucleotides coding for the same,
p) an aminopeptidase C (PepC) having a sequence identity of at least 90, 95 or 98%, preferably 100%, to the sequence according to SEQ ID NO: 138 and polynucleotides coding for the same,
q) a pyruvate dehydrogenase having a sequence identity of at least 90, 95 or 98%, preferably 100%, to the sequence according to SEQ ID NO: 140 and polynucleotides coding for the same,
r) a pyruvate kinase (pyruvate kinase 1) having a sequence identity of at least 90, 95 or 98%, preferably 100%, to the sequence according to SEQ ID NO: 142 and polynucleotides coding for the same,
s) a pyruvate kinase (pyruvate kinase 2) having a sequence identity of at least 90, 95 or 98%, preferably 100%, to the sequence according to SEQ ID NO: 144 and polynucleotides coding for the same,
t) an enolase having a sequence identity of at least 90, 95 or 98%, preferably 100%, to the sequence according to SEQ ID NO: 146 and polynucleotides coding for the same,
u) a 2,3-bisphosphoglycerate-dependent phosphoglycerate mutase (GpmA) having a sequence identity of at least 90, 95 or 98%, preferably 100%, to the sequence according to SEQ ID NO: 148 and polynucleotides coding for the same,
v) a phosphoglycerate kinase (Pgk) having a sequence identity of at least 90, 95 or 98%, preferably 100%, to the sequence according to SEQ ID NO: 150 and
w) a glyceraldehyde-3-phosphate dehydrogenase (glycerol-3-phosphate dehydrogenase 1) having a sequence identity of at least 90, 95 or 98%, preferably 100%, to the sequence according to SEQ ID NO: 152 and polynucleotides coding for the same,
x) a glyceraldehyde-3-phosphate dehydrogenase (glycerol-3-phosphate dehydrogenase 2) having a sequence identity of at least 90, 95 or 98%, preferably 100%, to the sequence according to SEQ ID NO: 154 and polynucleotides coding for the same,
y) a triosephosphate isomerase (TpiA) having a sequence identity of at least 90, 95 or 98%, preferably 100%, to the sequence according to SEQ ID NO: 156 and polynucleotides coding for the same,
z) a fructose bisphosphate aldolase having a sequence identity of at least 90, 95 or 98%, preferably 100%, to the sequence according to SEQ ID NO: 158 and polynucleotides coding for the same,
aa) a 1-phosphofructokinase having a sequence identity of at least 90, 95 or 98%, preferably 100%, to the sequence according to SEQ ID NO: 160 and polynucleotides coding for the same,
bb) a 6-phosphofructokinase having a sequence identity of at least 90, 95 or 98%, preferably 100%, to the sequence according to SEQ ID NO: 162 and polynucleotides coding for the same,
cc) a homoserine kinase (ThrB, EC 2.7.1.39) having a sequence identity of at least 90, 95 or 98%, preferably 100%, to the sequence according to SEQ ID NO: 4 and polynucleotides coding for the same,
dd) a cysteine synthase (CBS, CysK) having a sequence identity of at least 90, 95 or 98%, preferably 100%, to the sequence according to SEQ ID NO: 22 and polynucleotides coding for the same,
ee) a cystathionine beta-lyase (AecD) having a sequence identity of at least 90, 95 or 98%, preferably 100%, to the sequence according to SEQ ID NO: 26 and polynucleotides coding for the same,
ff) an aspartate semialdehyde dehydrogenase (Asd, EC 1.2.1.11) having a sequence identity of at least 90, 95 or 98%, preferably 100%, to the sequence according to SEQ ID NO: 28 and polynucleotides coding for the same,
gg) the smaller subunit of a transporter for branched-chain amino acids (BrnE) having a sequence identity of at least 90, 95 or 98%, preferably 100%, to the sequence according to SEQ ID NO: 30 and polynucleotides coding for the same,
hh) the larger subunit of a transporter for branched-chain amino acids (BrnF) having a sequence identity of at least 90, 95 or 98%, preferably 100%, to the sequence according to SEQ ID NO: 32 and polynucleotides coding for the same,
ii) a serine acetyltransferase (CysE) having a sequence identity of at least 90, 95 or 98%, preferably 100%, to the sequence according to SEQ ID NO: 34 and polynucleotides coding for the same,
jj) a cysteine synthase (CysK) having a sequence identity of at least 90, 95 or 98%, preferably 100%, to the sequence according to SEQ ID NO: 36 and polynucleotides coding for the same,
kk) a serine hydroxymethyltransferase (GlyA) having a sequence identity of at least 90, 95 or 98%, preferably 100%, to the sequence according to SEQ ID NO: 44 and polynucleotides coding for the same,
ll) an optionally feedback-resistant homoserine dehydrogenase (Hom, EC 1.2.1.11) having a sequence identity of at least 90, 95 or 98%, preferably 100%, to the sequence according to SEQ ID NO: 46 and polynucleotides coding for the same,
mm) an optionally feedback-resistant aspartate kinase (LysC, EC 2.7.2.4) having a sequence identity of at least 90, 95 or 98%, preferably 100%, to the sequence according to SEQ ID NO: 54 and polynucleotides coding for the same,
nn) a cystathionine gamma-synthase (MetB) having a sequence identity of at least 90, 95 or 98%, preferably 100%, to the sequence according to SEQ ID NO: 56 and polynucleotides coding for the same,
oo) a 5,10-methylenetetrahydrofolate reductase (MetF) having a sequence identity of at least 90, 95 or 98%, preferably 100%, to the sequence according to SEQ ID NO: 58 and polynucleotides coding for the same,
pp) a homoserine 0-acetyltransferase (MetX) having a sequence identity of at least 90, 95 or 98%, preferably 100%, to the sequence according to SEQ ID NO: 60 and polynucleotides coding for the same,
qq) an O-acetylhomoserine lyase (MetY) having a sequence identity of at least 90, 95 or 98%, preferably 100%, to the sequence according to SEQ ID NO: 62 and polynucleotides coding for the same,
rr) an optionally feedback-resistant pyruvate carboxylase (Pyc, EC 6.4.1.1) having a sequence identity of at least 90, 95 or 98%, preferably 100%, to the sequence according to SEQ ID NO: 64 and polynucleotides coding for the same,
ss) an optionally feedback-resistant D-3-phosphoglycerate dehydrogenase (SerA) having a sequence identity of at least 90, 95 or 98%, preferably 100%, to the sequence according to SEQ ID NO: 66 and polynucleotides coding for the same,
tt) a phosphoserine phosphatase (SerB) having a sequence identity of at least 90, 95 or 98%, preferably 100%, to the sequence according to SEQ ID NO: 68 and polynucleotides coding for the same,
uu) a phosphoserine aminotransferase (SerC) having a sequence identity of at least 90, 95 or 98%, preferably 100%, to the sequence according to SEQ ID NO: 70 and polynucleotides coding for the same,
vv) the subunit of a sulphate adenylyltransferase (CysD) having a sequence identity of at least 90, 95 or 98%, preferably 100%, to the sequence according to SEQ ID NO: 74 and polynucleotides coding for the same,
ww) an adenosine phosphosulphate reductase (CysH) having a sequence identity of at least 90, 95 or 98%, preferably 100%, to the sequence according to SEQ ID NO: 76 and polynucleotides coding for the same,
xx) a sulphite reductase (Cysl) having a sequence identity of at least 90, 95 or 98%, preferably 100%, to the sequence according to SEQ ID NO: 78 and polynucleotides coding for the same,
yy) an NADPH-dependent glutamate synthase beta chain (CysJ) having a sequence identity of at least 90, 95 or 98%, preferably 100%, to the sequence according to SEQ ID NO: 80 and polynucleotides coding for the same,
zz) the large subunit of a sulphate adenylyltransferase (CysN) having a sequence identity of at least 90, 95 or 98%, preferably 100%, to the sequence according to SEQ ID NO: 82 and polynucleotides coding for the same,
aaa) a cystathionine beta-synthase (CysY) having a sequence identity of at least 90, 95 or 98%, preferably 100%, to the sequence according to SEQ ID NO: 84 and polynucleotides coding for the same,
bbb) a sulphate transporter (CysZ) having a sequence identity of at least 90, 95 or 98%, preferably 100%, to the sequence according to SEQ ID NO: 86 and polynucleotides coding for the same,
ccc) a 5-methyltetrahydropteroyltriglutamate-homocysteine methyltransferase (MetE) having a sequence identity of at least 90, 95 or 98%, preferably 100%, to the sequence according to SEQ ID NO: 88 and polynucleotides coding for the same,
ddd) a peptidyl-tRNA hydrolase 1 (PtH1) having a sequence identity of at least 90, 95 or 98%, preferably 100%, to the sequence according to SEQ ID NO: 90 and polynucleotides coding for the same,
eee) a peptidyl-tRNA hydrolase 2 (PtH2) having a sequence identity of at least 90, 95 or 98%, preferably 100%, to the sequence according to SEQ ID NO: 92 and polynucleotides coding for the same,
fff) a diaminopimelate dehydrogenase (Ddh, EC 1.4.1.16) having a sequence identity of at least 90, 95 or 98%, preferably 100%, to the sequence according to SEQ ID NO: 202 and polynucleotides coding for the same,
ggg) a diaminopimelate decarboxylase (LysA, EC 4.1.1.20) having a sequence identity of at least 90, 95 or 98%, preferably 100%, to the sequence according to SEQ ID NO: 164 and polynucleotides coding for the same,
hhh) an aspartate aminotransferase (AaT, EC 2.6.1.1) having a sequence identity of at least 90, 95 or 98%, preferably 100%, to the sequence according to SEQ ID NO: 166 and polynucleotides coding for the same,
iii) an L-lysine exporter (LysE, lysine efflux permease) having a sequence identity of at least 90, 95 or 98%, preferably 100%, to the sequence according to SEQ ID NO: 168 and polynucleotides coding for the same,
jjj) a dihydropicolinate reductase (DapB, EC 1.3.1.26) having a sequence identity of at least 90, 95 or 98%, preferably 100%, to the sequence according to SEQ ID NO: 170 and polynucleotides coding for the same,
kkk) a glucose-6-phosphate dehydrogenase (EC 1.1.1.49) having a sequence identity of at least 90, 95 or 98%, preferably 100%, to the sequence according to SEQ ID NO: 172 and polynucleotides coding for the same,
lll) the Zwf subunit of a glucose-6-phosphate dehydrogenase (Zwf, EC 1.1.1.49) having a sequence identity of at least 90, 95 or 98%, preferably 100%, to the sequence according to SEQ ID NO: 186 and polynucleotides coding for the same,
mmm) the OpcA subunit of a glucose-6-phosphate dehydrogenase (OpcA, EC 1.1.1.49) having a sequence identity of at least 90, 95 or 98%, preferably 100%, to the sequence according to SEQ ID NO: 188 and polynucleotides coding for the same,
nnn) a phosphogluconic acid dehydrogenase (Gnd, EC 1.1.1.44) having a sequence identity of at least 90, 95 or 98%, preferably 100%, to the sequence according to SEQ ID NO: 174 and polynucleotides coding for the same.

The present invention further relates also to vectors comprising the polynucleotides polynucleotides and/or vectors mentioned above. In a preferred embodiment, the relevant polypeptide and/or polynucleotide is present in this case in the microorganism in overexpressed form. The recombinant microorganisms are preferably in this case coryneform bacteria, especially Corynebacteria, particularly those of the species C. humireducens or C. glutamicum.

The present invention therefore further relates also to a method for overproducing an L-amino acid, preferably selected from L-alanine, L-valine, L-amino acids of the glutamate family, particularly L-glutamate, L-glutamine, L-proline and L-arginine, and L-amino acids of the aspartate family, particularly L-aspartate, L-asparagine, L-methionine, L-lysine, L-isoleucine and L-threonine, particularly preferably selected from L-alanine, L-valine, L-glutamate, L-methionine, L-lysine and L-threonine, especially for overproducing L-methionine, in which at least one, preferably at least two, three or four, of the polynucleotides mentioned are present in overexpressed form, wherein the method is preferably carried out in Corynebacteria, particularly those of the species C. humireducens or C. glutamicum.

The present invention further relates also to other polynucleotides from C. humireducens and also the polypeptides encoded by said polynucleotides. By means of deactivation or attenuation of the relevant polynucleotides or polypeptides, the amino acid production of certain L-amino acids can be positively influenced.

The present invention therefore also relates to polypeptides selected from the following list:
a) a threonine synthase (ThrC, EC 4.2.3.1) having a sequence identity of at least 90, 95 or 98%, preferably 100%, to the sequence according to SEQ ID NO: 108 and polynucleotides coding for the same,
b) an isopropylmalate synthase (LeuA, EC 2.3.3.13) having a sequence identity of at least 90, 95 or 98%, preferably 100%, to the sequence according to SEQ ID NO: 110 and polynucleotides coding for the same,
c) an isopropylmalate dehydrogenase (LeuB, EC 1.1.1.85) having a sequence identity of at least 90, 95 or 98%, preferably 100%, to the sequence according to SEQ ID NO: 112 and polynucleotides coding for the same,
d) the subunits of an isopropylmalate isomerase (LeuCD, EC 4.2.1.33) having sequence identities of at least 90, 95 or 98%, preferably 100%, to the sequences according to SEQ ID NO: 114 or SEQ ID NO: 116 and polynucleotides coding for the same,
e) the subunits of a succinyl-CoA ligase (SucCD, EC 6.2.1.5) each having sequence identities of at least 90, 95 or 98%, preferably 100%, to the sequences according to SEQ ID NO: 198 or SEQ ID NO: 200 and polynucleotides coding for the same,
f) a DNA binding domain of type HTH tetR (McbR) having a sequence identity of at least 90, 95 or 98%, preferably 100%, to the sequence according to SEQ ID NO: 2 and polynucleotides coding for the same,
g) a homoserine kinase (ThrB, EC 2.7.1.39) having a sequence identity of at least 90, 95 or 98%, preferably 100%, to the sequence according to SEQ ID NO: 4 and polynucleotides coding for the same,
h) a glucose-6-phosphate isomerase (Pgi, EC 5.3.1.9) having a sequence identity of at least 90, 95 or 98%, preferably 100%, to the sequence according to SEQ ID NO: 6 and polynucleotides coding for the same,
i) a phosphoenolpyruvate carboxykinase (Pck, EC 4.1.1.32) having a sequence identity of at least 90, 95 or 98%, preferably 100%, to the sequence according to SEQ ID NO: 8 and polynucleotides coding for the same,
j) a D-methionine-binding lipoprotein (MetQ) having a sequence identity of at least 90, 95 or 98%, preferably 100%, to the sequence according to SEQ ID NO: 10 and polynucleotides coding for the same,
k) a methionine transporter (MetP) having a sequence identity of at least 90, 95 or 98%, preferably 100%, to the sequence according to SEQ ID NO: 12 and polynucleotides coding for the same,
l) an ATP-dependent methionine transporter (MetN) having a sequence identity of at least 90, 95 or 98%, preferably 100%, to the sequence according to SEQ ID NO: 14 and polynucleotides coding for the same,
m) an S-adenosylmethionine synthase (MetK) having a sequence identity of at least 90, 95 or 98%, preferably 100%, to the sequence according to SEQ ID NO: 16 and polynucleotides coding for the same,
n) a methionine import system permease (MetI) having a sequence identity of at least 90, 95 or 98%, preferably 100%, to the sequence according to SEQ ID NO: 18 and polynucleotides coding for the same,
o) a 4-hydroxy-tetrahydrodipicolinate synthase (DapA, EC 4.3.3.7) having a sequence identity of at least 90, 95 or 98%, preferably 100%, to the sequence according to SEQ ID NO: 20 and polynucleotides coding for the same,
p) a carboxylate-amine ligase having a sequence identity of at least 90, 95 or 98%, preferably 100%, to the sequence according to SEQ ID NO: 24 and polynucleotides coding for the same,
q) a malate:quinone oxidoreductase (Mqo, EC 1.1.99.16) having a sequence identity of at least 90, 95 or 98%, preferably 100%, to the sequence according to SEQ ID NO: 176 and polynucleotides coding for the same,
r) the E1p subunit of a pyruvate dehydrogenase complex (AceE, EC 1.2.4.1) having a sequence identity of at least 90, 95 or 98%, preferably 100%, to the sequence according to SEQ ID NO: 178 and polynucleotides coding for the same,
s) a citrate synthase (GltA, EC 4.1.3.7) having a sequence identity of at least 90, 95 or 98%, preferably 100%, to the sequence according to SEQ ID NO: 180 and polynucleotides coding for the same,
t) a malate dehydrogenase (Mdh, EC 1.1.1.37) having a sequence identity of at least 90, 95 or 98%, preferably 100%, to the sequence according to SEQ ID NO: 182 and polynucleotides coding for the same,
u) a UDP-N-acetylmuramoylalanyl-D-glutamate-2,6-diaminopimelate ligase (MurE, EC 6.3.2.13) having a sequence identity of at least 90, 95 or 98%, preferably 100%, to the sequence according to SEQ ID NO: 184, and polynucleotides coding for the same,

The present invention further relates also to vectors comprising the polynucleotides mentioned above and also recombinant microorganisms comprising the enzymes and/or polynucleotides and/or vectors mentioned above. In a preferred embodiment, the relevant polypeptide and/or polynucleotide is present in this case in the microorganism in deactivated or attenuated form. The recombinant microorganisms are preferably in this case coryneform bacteria, especially Corynebacteria, particularly those of the species C. humireducens or C. glutamicum, especially of the species C. humireducens.

The present invention therefore further relates also to a method for overproducing an L-amino acid, preferably selected from L-alanine, L-valine, L-amino acids of the glutamate family, particularly L-glutamate, L-glutamine, L-proline and L-arginine, and L-amino acids of the aspartate family, particularly L-aspartate, L-asparagine, L-methionine, L-lysine, L-isoleucine and L-threonine, particularly preferably selected from L-alanine, L-valine, L-glutamate, L-methionine, L-lysine and L-threonine, especially for overproducing L-methionine, in which at least one, preferably at least two, three or four, of the polynucleotides mentioned are present in deactivated or attenuated form, wherein the method is preferably carried out in Corynebacteria, particularly those of the species C. humireducens or C. glutamicum. In a preferred embodiment, at least one, preferably at least two, three or four of the polynucleotides mentioned in the detailed list above is present in this case at the same time in overexpressed form.

In a further preferred embodiment according to the invention, microorganisms or bacteria according to the invention, particularly Corynebacteria according to the invention, especially Corynebacteria according to the invention of the species C. humireducens or C. glutamicum, particularly L-methionine-overproducing strains according to the invention, in addition to an inventive, preferably overexpressed glycine cleavage system or polynucleotides coding for said system, have at least one, preferably at least two or three, particularly preferably at least four or five, of the following features:
a) an attenuated polynucleotide (mcbR), which codes for a DNA binding domain of the type HTH tetR (McbR), preferably for a DNA binding domain having a sequence identity of at least 90, 95 or 98%, preferably 100%, to the sequence according to SEQ ID NO: 2,
b) an attenuated polynucleotide (thrB gene), which codes for a homoserine kinase (ThrB, EC 2.7.1.39), preferably for a homoserine kinase having a sequence identity of at least 90, 95 or 98%, preferably 100%, to the sequence according to SEQ ID NO: 4,
c) an attenuated polynucleotide (pgi), which codes for a glucose-6-phosphate isomerase (Pgi, EC 5.3.1.9), preferably for a glucose-6-phosphate isomerase having a sequence identity of at least 90, 95 or 98%, preferably 100%, to the sequence according to SEQ ID NO: 6,
d) an attenuated polynucleotide (pck), which codes for a phosphoenolpyruvate carboxykinase (Pck, EC 4.1.1.32), preferably for a phosphoenolpyruvate carboxykinase having a sequence identity of at least 90, 95 or 98%, preferably 100%, to the sequence according to SEQ ID NO: 8,
e) an attenuated polynucleotide (metQ), which codes for a D-methionine-binding lipoprotein (MetQ), preferably for a D-methionine-binding lipoprotein having a sequence identity of at least 90, 95 or 98%, preferably 100%, to the sequence according to SEQ ID NO: 10,
f) an attenuated polynucleotide (metP), which codes for a methionine transporter (MetP), preferably for a methionine transporter having a sequence identity of at least 90, 95 or 98%, preferably 100%, to the sequence according to SEQ ID NO: 12,
g) an attenuated polynucleotide (metN), which codes for an ATP-dependent methionine transporter (MetN), preferably for an ATP-dependent methionine transporter having a sequence identity of at least 90, 95 or 98%, preferably 100%, to the sequence according to SEQ ID NO: 14,
h) an attenuated polynucleotide (metK), which codes for an S-adenosylmethionine synthase (MetK), preferably for an S-adenosylmethionine synthase having a sequence identity of at least 90, 95 or 98%, preferably 100%, to the sequence according to SEQ ID NO: 16,
i) an attenuated polynucleotide (metI), which codes for a methionine import system permease (MetI), preferably for a methionine import system permease having a sequence identity of at least 90, 95 or 98%, preferably 100%, to the sequence according to SEQ ID NO: 18,
j) an attenuated polynucleotide (dapA), which codes for a 4-hydroxy-tetrahydrodipicolinate synthase (DapA), preferably for a 4-hydroxy-tetrahydrodipicolinate synthase having a sequence identity of at least 90, 95 or 98%, preferably 100%, to the sequence according to SEQ ID NO: 20,
k) an overexpressed polynucleotide (CBS), which codes for a cysteine synthase (CBS), preferably for a cysteine synthase having a sequence identity of at least 90, 95 or 98%, preferably 100%, to the sequence according to SEQ ID NO: 22,
l) an attenuated polynucleotide, which codes for a cg3031 homologue, preferably for a cg3031 homologue having a sequence identity of at least 90, 95 or 98%, preferably 100%, to the sequence according to SEQ ID NO: 24,
m) an overexpressed polynucleotide (aecD), which codes for a cystathionine beta-lyase (AecD), preferably for a cystathionine beta-lyase having a sequence identity of at least 90, 95 or 98%, preferably 100%, to the sequence according to SEQ ID NO: 26,
n) an overexpressed polynucleotide (asd), which codes for an aspartate semialdehyde dehydrogenase (Asd), preferably for an aspartate semialdehyde dehydrogenase having a sequence identity of at least 90, 95 or 98%, preferably 100%, to the sequence according to SEQ ID NO: 28,
o) an overexpressed polynucleotide (metH), which codes for a 5-methyltetrahydrofolate homocysteine methyltransferase (MetH, EC 2.1.1.13),
p) an overexpressed polynucleotide (brnE), which codes for the smaller subunit of a transporter for branched-chain amino acids (BrnE), preferably for a subunit having a sequence identity of at least 90, 95 or 98%, preferably 100%, to the sequence
q) an overexpressed polynucleotide (brnF), which codes for the larger subunit of a transporter for branched-chain amino acids (BrnF), preferably for a subunit having a sequence identity of at least 90, 95 or 98%, preferably 100%, to the sequence according to SEQ ID NO: 32,
r) an overexpressed polynucleotide (cysE), which codes for a serine acetyltransferase (CysE), preferably for a serine acetyltransferase having a sequence identity of at least 90, 95 or 98%, preferably 100%, to the sequence according to SEQ ID NO: 34,
s) an overexpressed polynucleotide (cysK), which codes for a cysteine synthase (CysK), preferably for a cysteine synthase having a sequence identity of at least 90, 95 or 98%, preferably 100%, to the sequence according to SEQ ID NO: 36,
t) an overexpressed polynucleotide (glyA), which codes for a serine hydroxymethyltransferase (GlyA), preferably for a serine hydroxymethyltransferase having a sequence identity of at least 90, 95 or 98%, preferably 100%, to the sequence according to SEQ ID NO: 44,
u) an overexpressed polynucleotide (hom), which codes for an optionally feedback-resistant homoserine dehydrogenase (Hom), preferably for a homoserine dehydrogenase having a sequence identity of at least 90, 95 or 98%, preferably 100%, to the sequence according to SEQ ID NO: 46,
v) an overexpressed polynucleotide (lysC), which codes for an optionally feedback-resistant aspartate kinase (LysC), preferably for an aspartate kinase having a sequence identity of at least 90, 95 or 98%, preferably 100%, to the sequence according to SEQ ID NO: 54,
w) an overexpressed polynucleotide (metB), which codes for a cystathionine gamma-synthase (MetB), preferably for a cystathionine gamma-synthase having a sequence identity of at least 90, 95 or 98%, preferably 100%, to the sequence according to SEQ ID NO: 56,
x) an overexpressed polynucleotide (metF), which codes for a 5,10-methylenetetrahydrofolate reductase (MetF), preferably for a 5,10-methylenetetrahydrofolate reductase having a sequence identity of at least 90, 95 or 98%, preferably 100%, to the sequence according to SEQ ID NO: 58,
y) an overexpressed polynucleotide (metX), which codes for a homoserine 0-acetyltransferase (MetX), preferably for a homoserine 0-acetyltransferase having a sequence identity of at least 90, 95 or 98%, preferably 100%, to the sequence according to SEQ ID NO: 60,
z) an overexpressed polynucleotide (metY), which codes for an O-acetylhomoserine lyase (MetY), preferably for an O-acetylhomoserine lyase having a sequence identity of at least 90, 95 or 98%, preferably 100%, to the sequence according to SEQ ID NO: 62,
aa) an overexpressed polynucleotide (pyc), which codes for a pyruvate carboxylase (Pyc), preferably for a pyruvate carboxylase having a sequence identity of at least 90, 95 or 98%, preferably 100%, to the sequence according to SEQ ID NO: 64,
bb) an overexpressed polynucleotide (serA), which codes for an optionally feedback-resistant D-3-phosphoglycerate dehydrogenase (SerA), preferably for a D-3-phosphoglycerate dehydrogenase having a sequence identity of at least 90, 95 or 98%, preferably 100%, to the sequence according to SEQ ID NO: 66,
cc) an overexpressed polynucleotide (serB), which codes for a phosphoserine phosphatase (SerB), preferably for a phosphoserine phosphatase having a sequence identity of at least 90, 95 or 98%, preferably 100%, to the sequence according to SEQ ID NO: 68,
dd) an overexpressed polynucleotide (serC), which codes for a phosphoserine aminotransferase (SerC), preferably for a phosphoserine aminotransferase having a sequence identity of at least 90, 95 or 98%, preferably 100%, to the sequence according to SEQ ID NO: 70,
ee) an overexpressed polynucleotide (ald), which codes for an alanine dehydrogenase (Ald), preferably for an alanine dehydrogenase having a sequence identity of at least 90, 95 or 98%, preferably 100%, to the sequence according to SEQ ID NO: 72,
ff) an overexpressed polynucleotide (cysD), which codes for the subunit of a sulphate adenylyltransferase (CysD), preferably for a subunit having a sequence identity of at least 90, 95 or 98%, preferably 100%, to the sequence according to SEQ ID NO: 74,
gg) an overexpressed polynucleotide (cysH), which codes for an adenosine phosphosulphate reductase (CysH), preferably for an adenosine phosphosulphate reductase having a sequence identity of at least 90, 95 or 98%, preferably 100%, to the sequence according to SEQ ID NO: 76,
hh) an overexpressed polynucleotide (cysl), which codes for a sulphite reductase (Cysl), preferably for a sulphite reductase having a sequence identity of at least 90, 95 or 98%, preferably 100%, to the sequence according to SEQ ID NO: 78,
ii) an overexpressed polynucleotide (cysJ), which codes for (CysJ), preferably for one having a sequence identity of at least 90, 95 or 98%, preferably 100%, to the sequence according to SEQ ID NO: 80,
jj) an overexpressed polynucleotide (cysN), which codes for the subunit of a sulphate adenylyltransferase (CysN), preferably for a subunit having a sequence identity of at least 90, 95 or 98%, preferably 100%, to the sequence according to SEQ ID NO: 82,
kk) an overexpressed polynucleotide (cysY), which codes for a cystathionine beta-synthase (CysY), preferably for a cystathionine beta-synthase having a sequence identity of at least 90, 95 or 98%, preferably 100%, to the sequence according to SEQ ID NO: 84,
ll) an overexpressed polynucleotide (cysZ), which codes for a putative sulphate transporter (CysZ), preferably for a sulphate transporter having a sequence identity of at least 90, 95 or 98%, preferably 100%, to the sequence according to SEQ ID NO: 86,
mm) an overexpressed polynucleotide (metE), which codes for a 5-methyltetrahydropteroyltriglutamate-homocysteine methyltransferase (MetE), preferably for a protein having a sequence identity of at least 90, 95 or 98%, preferably 100%, to the sequence according to SEQ ID NO: 88,
nn) an overexpressed polynucleotide (ptH1), which codes for a peptidyl-tRNA hydrolase 1 (PtH1), preferably for a peptidyl-tRNA hydrolase 1 having a sequence identity of at least 90, 95 or 98%, preferably 100%, to the sequence according to SEQ ID NO: 90,
oo) an overexpressed polynucleotide (ptH2), which codes for a peptidyl-tRNA hydrolase 2 (PtH2), preferably for a peptidyl-tRNA hydrolase 2 having a sequence identity of at least 90, 95 or 98%, preferably 100%, to the sequence according to SEQ ID NO: 92.

The present invention further relates also to a corresponding method for overproducing an L-amino acid, particularly L-methionine, in which such a microorganism or such a bacterium is used.

In a further preferred embodiment according to the invention, microorganisms or bacteria according to the invention, particularly Corynebacteria according to the invention, especially Corynebacteria according to the invention of the species C. humireducens or C. glutamicum, particularly L-valine-overproducing strains according to the invention, in addition to an inventive, preferably overexpressed glycine cleavage system or polynucleotides coding for said system, have at least one, preferably at least 2 or 3, particularly preferably at least 4 or 5, of the following features:
a) an overexpressed polynucleotide (ilvA gene), which codes for a threonine dehydratase (llvA EC 4.3.1.19), preferably for a threonine dehydratase having a sequence identity of at least 90, 95 or 98%, preferably 100%, to the sequence according to SEQ ID NO: 106,
b) an overexpressed polynucleotide (ilvB gene), which codes for the subunit of an acetolactate synthase (llvB), preferably for the subunit of an acetolactate synthase having a sequence identity of at least 90, 95 or 98%, preferably 100%, to the sequence according to SEQ ID NO: 98,
c) an overexpressed polynucleotide (ilvN gene), which codes for the optionally feedback-resistant subunit of an acetolactate synthase (llvN),
d) an overexpressed polynucleotide (ilvC gene), which codes for an isomeroreductase (llvC, EC 1.1.1.86), preferably for an isomeroreductase having a sequence identity of at least 90, 95 or 98%, preferably 100%, to the sequence according to SEQ ID NO: 100,
e) an overexpressed polynucleotide (ilvD gene), which codes for a dihydroxyacid dehydratase (llvD, EC 4.2.1.9), preferably for a dihydroxyacid dehydratase having a sequence identity of at least 90, 95 or 98%, preferably 100%, to the sequence according to SEQ ID NO: 102,
f) an overexpressed polynucleotide (ilvE gene), which codes for a transaminase (llvE, EC 2.6.1.42), preferably for a transaminase having a sequence identity of at least 90, 95 or 98%, preferably 100%, to the sequence according to SEQ ID NO: 104,
g) an overexpressed polynucleotide (ilvH gene), which codes for an acetolactate synthase (llvH, EC 2.2.1.6), preferably for an acetolactate synthase having a sequence identity of at least 90, 95 or 98%, preferably 100%, to the sequence according to SEQ ID NO: 122,
h) an attenuated polynucleotide (thrB gene), which codes for a homoserine kinase (ThrB, EC 2.7.1.39), preferably for a homoserine kinase having a sequence identity of at least 90, 95 or 98%, preferably 100%, to the sequence according to SEQ ID NO: 4,
i) an attenuated polynucleotide (thrC gene), which codes for a threonine synthase (ThrC, EC 4.2.3.1), preferably for a threonine synthase having a sequence identity of at least 90, 95 or 98%, preferably 100%, to the sequence according to SEQ ID NO: 108,
j) an overexpressed polynucleotide (horn gene), which codes for an optionally feedback-resistant homoserine dehydrogenase (Horn, EC 1.2.1.11), preferably for a homoserine dehydrogenase having a sequence identity of at least 90, 95 or 98%, preferably 100%, to the sequence according to SEQ ID NO: 46,
k) an attenuated polynucleotide (leuA gene), which codes for an optionally feedback-resistant isopropylmalate synthase (LeuA, EC 2.3.3.13), preferably for an isopropylmalate synthase having a sequence identity of at least 90, 95 or 98%, preferably 100%, to the sequence according to SEQ ID NO: 110,
I) an attenuated polynucleotide (leuB gene), which codes for an isopropylmalate dehydrogenase (LeuB, EC 1.1.1.85), preferably for an isopropylmalate dehydrogenase having a sequence identity of at least 90, 95 or 98%, preferably 100%, to the sequence according to SEQ ID NO: 112,
m) attenuated polynucleotides (leuCD gene), which code for the subunits of an isopropylmalate isomerase (LeuCD, EC 4.2.1.33), preferably for the subunits of an isopropylmalate isomerase having sequence identities of at least 90, 95 or 98%, preferably 100%, to the sequences according to SEQ ID NO: 114 and SEQ ID NO: 116,
n) an overexpressed polynucleotide (panB gene), which codes for a 3-methyl-2-oxobutanoate hydroxymethyltransferase (PanB, EC 2.1.2.11), preferably for a 3-methyl-2-oxobutanoate hydroxymethyltransferase having a sequence identity of at least 90, 95 or 98%, preferably 100%, to the sequence according to SEQ ID NO: 118,
o) an overexpressed polynucleotide (panC gene), which codes for a pantothenate synthase (PanC, EC 6.3.2.1), preferably for a pantothenate synthase having a sequence identity of at least 90, 95 or 98%, preferably 100%, to the sequence according to SEQ ID NO: 120.

The present invention further relates accordingly also to a method for overproducing an L-amino acid, particularly L-valine, in which such a microorganism or such a bacterium is used.

In a further preferred embodiment according to the invention, microorganisms or bacteria according to the invention, particularly Corynebacteria according to the invention, especially Corynebacteria according to the invention of the species C. humireducens or C. glutamicum, particularly L-glutamate-overproducing strains according to the invention, in addition to an inventive, preferably overexpressed glycine cleavage system or polynucleotides coding for said system, have at least one, preferably at least two or three, particularly preferably at least four or five, of the following features:
a) an overexpressed polynucleotide (gdh), which codes for a glutamate dehydrogenase (Gdh), preferably for a glutamate dehydrogenase having a sequence identity of at least 90, 95 or 98%, preferably 100%, to the sequence according to SEQ ID NO: 124,
b) an overexpressed polynucleotide, which codes for a glutamine synthetase (glutamine synthetase 1), preferably for a glutamine synthetase having a sequence identity of at least 90, 95 or 98%, preferably 100%, to the sequence according to SEQ ID NO: 126,
c) an overexpressed polynucleotide, which codes for a glutamine synthetase (glutamine synthetase 2), preferably for a glutamine synthetase having a sequence identity of at least 90, 95 or 98%, preferably 100%, to the sequence according to SEQ ID NO: 128,
d) an overexpressed polynucleotide, which codes for a glutamate synthase, preferably for a glutamate synthase having a sequence identity of at least 90, 95 or 98%, preferably 100%, to the sequence according to SEQ ID NO: 130,
e) an overexpressed polynucleotide, which codes for an isocitrate dehydrogenase, preferably for an isocitrate dehydrogenase having a sequence identity of at least 90, 95 or 98%, preferably 100%, to the sequence according to SEQ ID NO: 132,
f) an overexpressed polynucleotide, which codes for an aconitate hydrase, preferably for an aconitate hydrase having a sequence identity of at least 90, 95 or 98%, preferably 100%, to the sequence according to SEQ ID NO: 134,
g) an overexpressed polynucleotide, which codes for a citrate synthase, preferably for a citrate synthase having a sequence identity of at least 90, 95 or 98%, preferably 100%, to the sequence according to SEQ ID NO: 136,
h) an overexpressed polynucleotide (pepC), which codes for an aminopeptidase C (PepC), preferably for an aminopeptidase C having a sequence identity of at least 90, 95 or 98%, preferably 100%, to the sequence according to SEQ ID NO: 138,
i) an overexpressed polynucleotide, which codes for a pyruvate dehydrogenase, preferably for a pyruvate dehydrogenase having a sequence identity of at least 90, 95 or 98%, preferably 100%, to the sequence according to SEQ ID NO: 140,
j) an overexpressed polynucleotide, which codes for a pyruvate kinase (pyruvate kinase 1), preferably for a pyruvate kinase having a sequence identity of at least 90, 95 or 98%, preferably 100%, to the sequence according to SEQ ID NO: 142,
k) an overexpressed polynucleotide, which codes for a pyruvate kinase (pyruvate kinase 2), preferably for a pyruvate kinase having a sequence identity of at least 90, 95 or 98%, preferably 100%, to the sequence according to SEQ ID NO: 144,
l) an overexpressed polynucleotide, which codes for an enolase, preferably for an enolase having a sequence identity of at least 90, 95 or 98%, preferably 100%, to the sequence according to SEQ ID NO: 146,
m) an overexpressed polynucleotide (gpmA), which codes for a 2,3-bisphosphoglycerate-having a sequence identity of at least 90, 95 or 98%, preferably 100%, to the sequence according to SEQ ID NO: 148,
n) an overexpressed polynucleotide (pgk), which codes for a phosphoglycerate kinase (Pgk), preferably for a phosphoglycerate kinase having a sequence identity of at least 90, 95 or 98%, preferably 100%, to the sequence according to SEQ ID NO: 150,
o) an overexpressed polynucleotide, which codes for a glyceraldehyde-3-phosphate dehydrogenase (glycerol-3-phosphate dehydrogenase 1), preferably for a glyceraldehyde-3-phosphate dehydrogenase having a sequence identity of at least 90, 95 or 98%, preferably 100%, to the sequence according to SEQ ID NO: 152,
p) an overexpressed polynucleotide, which codes for a glyceraldehyde-3-phosphate dehydrogenase (glycerol-3-phosphate dehydrogenase 2), preferably for a glyceraldehyde-3-phosphate dehydrogenase having a sequence identity of at least 90, 95 or 98%, preferably 100%, to the sequence according to SEQ ID NO: 154,
q) an overexpressed polynucleotide (tpiA), which codes for a triosephosphate isomerase (TpiA), preferably for a triosephosphate isomerase having a sequence identity of at least 90, 95 or 98%, preferably 100%, to the sequence according to SEQ ID NO: 156,
r) an overexpressed polynucleotide, which codes for a fructose bisphosphate aldolase, preferably for a fructose bisphosphate aldolase having a sequence identity of at least 90, 95 or 98%, preferably 100%, to the sequence according to SEQ ID NO: 158,
s) an overexpressed polynucleotide, which codes for a 1-phosphofructokinase, preferably for a 1-phosphofructokinase having a sequence identity of at least 90, 95 or 98%, preferably 100%, to the sequence according to SEQ ID NO: 160,
t) an overexpressed polynucleotide, which codes for a 6-phosphofructokinase, preferably for a 6-phosphofructokinase having a sequence identity of at least 90, 95 or 98%, preferably 100%, to the sequence according to SEQ ID NO: 162,
u) an overexpressed polynucleotide (pgi), which codes for a glucose-6-phosphate isomerase, preferably for a glucose-6-phosphate isomerase having a sequence identity of at least 90, 95 or 98%, preferably 100%, to the sequence according to SEQ ID NO: 6,
v) attenuated polynucleotides (sucCD), which code for the subunits of a succinyl-CoA ligase (SucCD, EC 6.2.1.5), preferably for the subunits of a succinyl-CoA ligase having sequence identities of at least 90, 95 or 98%, preferably 100%, to the sequences according to SEQ ID NO: 198 or SEQ ID NO: 200.

The present invention further relates accordingly also to a method for overproducing an L-amino acid, particularly L-glutamate, in which such a microorganism or such a bacterium is used.

In a further preferred embodiment according to the invention, microorganisms or bacteria according to the invention, particularly Corynebacteria according to the invention, especially Corynebacteria according to the invention of the species C. humireducens or C. glutamicum, particularly L-alanine-overproducing strains according to the invention, in addition to an inventive, preferably overexpressed glycine cleavage system or polynucleotides coding for said system, have at least one, preferably at least two or three, particularly preferably at least four or five, of the following features:
a) an overexpressed polynucleotide (alaD), which codes for an alanine dehydrogenase (AlaD), preferably for an alanine dehydrogenase from Corynebacteria,
b) an overexpressed polynucleotide (gapA), which codes for a glyceraldehyde-3-phosphate dehydrogenase (GapA), preferably for a glyceraldehyde-3-phosphate dehydrogenase from Corynebacteria,
c) a deactivated or attenuated polynucleotide (ldhA), which codes for an L-lactate dehydrogenase (LdhA), preferably for an L-lactate dehydrogenase from Corynebacteria,
d) a deactivated or attenuated polynucleotide (ppc), which codes for a phosphoenolpyruvate carboxylase (Ppc), preferably for a phosphoenolpyruvate carboxylase from Corynebacteria,
e) a deactivated or attenuated polynucleotide (alr), which codes for an alanine racemase (Alr), preferably for an alanine racemase from Corynebacteria.

The present invention further relates accordingly also to a method for overproducing an L-amino acid, particularly L-alanine, in which such a microorganism or such a bacterium is used.

In a further preferred embodiment according to the invention, microorganisms or bacteria according to the invention, particularly Corynebacteria according to the invention, especially Corynebacteria according to the invention of the species C. humireducens or C. glutamicum, particularly L-lysine-overproducing strains according to the invention, in addition to an inventive, preferably overexpressed glycine cleavage system or polynucleotides coding for said system, have at least one, preferably at least 2 or 3, particularly preferably at least 4 or 5, of the following features:
a) an overexpressed polynucleotide (dapA gene), which codes for a dihydrodipicolinate synthase (DapA, EC 4.2.1.52), preferably for a dihydrodipicolinate synthase having a sequence identity of at least 90, 95 or 98%, preferably 100%, to the sequence according to SEQ ID NO: 20,
b) an overexpressed polynucleotide (lysC), which codes for a preferably feedback-resistant aspartate kinase (LysC, EC 2.7.2.4), preferably for an aspartate kinase having a sequence identity of at least 90, 95 or 98%, preferably 100%, to the sequence according to SEQ ID NO: 54,
c) an overexpressed polynucleotide (ddh), which codes for a diaminopimelate dehydrogenase (Ddh, EC 1.4.1.16), preferably for a diaminopimelate dehydrogenase having a sequence identity of at least 90, 95 or 98%, preferably 100%, to the sequence according to SEQ ID NO: 202,
d) an overexpressed polynucleotide (asd gene), which codes for an aspartate semialdehyde dehydrogenase (Asd, EC 1.2.1.11), preferably for an aspartate semialdehyde dehydrogenase having a sequence identity of at least 90, 95 or 98%, preferably 100%, to the sequence according to SEQ ID NO: 28,
e) an overexpressed polynucleotide (lysA gene), which codes for a diaminopimelate decarboxylase (LysA, EC 4.1.1.20), preferably for a diaminopimelate decarboxylase having a sequence identity of at least 90, 95 or 98%, preferably 100%, to the sequence according to SEQ ID NO: 164,
f) an overexpressed polynucleotide (aat gene), which codes for an aspartate aminotransferase (AaT, EC 2.6.1.1), preferably for an aspartate aminotransferase having a sequence identity of at least 90, 95 or 98%, preferably 100%, to the sequence according to SEQ ID NO: 166,
g) an overexpressed polynucleotide (lysE gene), which codes for an L-lysine exporter (LysE, lysine efflux permease), preferably for an L-lysine exporter having a sequence identity of at least 90, 95 or 98%, preferably 100%, to the sequence according to SEQ ID NO: 168,
h) an overexpressed polynucleotide (pyc gene), which codes for a pyruvate carboxylase (Pyc, EC 6.4.1.1), preferably for a pyruvate carboxylase having a sequence identity of at least 90, 95 or 98%, preferably 100%, to the sequence according to SEQ ID NO: 64,
i) an overexpressed polynucleotide (dapF gene), which codes for a diaminopimelate epimerase (DapF, EC 5.1.1.7),
j) an overexpressed polynucleotide (dapB gene), which codes for a dihydropicolinate reductase (DapB, EC 1.3.1.26), preferably for a dihydropicolinate reductase having a sequence identity of at least 90, 95 or 98%, preferably 100%, to the sequence according to SEQ ID NO: 172,
k) an overexpressed polynucleotide, which codes for a glucose-6-phosphate dehydrogenase (EC 1.1.1.49), preferably for a glucose-6-phosphate dehydrogenase having a sequence identity of at least 90, 95 or 98%, preferably 100%, to the sequence according to SEQ ID NO: 174,
l) an overexpressed polynucleotide (zwf gene), which codes for a Zwf subunit of a glucose-6-phosphate dehydrogenase (Zwf, EC 1.1.1.49), preferably for a Zwf subunit having a sequence identity of at least 90, 95 or 98%, preferably 100%, to the sequence according to SEQ ID NO: 188,
m) an overexpressed polynucleotide (opcA gene), which codes for the OpcA subunit of a glucose-6-phosphate dehydrogenase (OpcA, EC 1.1.1.49), preferably for an OpcA subunit having a sequence identity of at least 90, 95 or 98%, preferably 100%, to the sequence according to SEQ ID NO: 190,
n) an overexpressed polynucleotide (gnd gene), which codes for a phosphogluconic acid dehydrogenase (Gnd, EC 1.1.1.44), preferably for a phosphogluconic acid dehydrogenase having a sequence identity of at least 90, 95 or 98%, preferably 100%, to the sequence according to SEQ ID NO: 176,
o) a deactivated or attenuated polynucleotide (mqo), which codes for a malate:quinone oxidoreductase (Mqo, EC 1.1.99.16), preferably for a malate:quinone oxidoreductase having a sequence identity of at least 90, 95 or 98%, preferably 100%, to the sequence according to SEQ ID NO: 178,
p) a deactivated or attenuated polynucleotide (aceE), which codes for the E1p subunit of a pyruvate dehydrogenase complex (AceE, EC 1.2.4.1), preferably for an E1p subunit having a sequence identity of at least 90, 95 or 98%, preferably 100%, to the sequence according to SEQ ID NO: 180,
q) a deactivated or attenuated polynucleotide (gltA), which codes for a citrate synthase (GltA, EC 4.1.3.7), preferably for a citrate synthase having a sequence identity of at least 90, 95 or 98%, preferably 100%, to the sequence according to SEQ ID NO: 182,
r) a deactivated or attenuated polynucleotide (mdh), which codes for a malate dehydrogenase (Mdh, EC 1.1.1.37), preferably for a malate dehydrogenase having a sequence identity of at least 90, 95 or 98%, preferably 100%, to the sequence according to SEQ ID NO: 184,
s) a deactivated or attenuated polynucleotide (murE), which codes for a UDP-N-acetylmuramoylalanyl-D-glutamate-2,6-diaminopimelate ligase (MurE, EC 6.3.2.13), preferably for an enzyme having a sequence identity of at least 90, 95 or 98%, preferably 100%, to the sequence according to SEQ ID NO: 186.

The present invention further relates accordingly also to a method for overproducing an L-amino acid, particularly L-lysine, in which such a microorganism or such a bacterium is used.

The polynucleotides and polypeptides used or to be used in the method according to the invention mentioned above preferably originate from Corynebacteria, particularly from C. glutamicum or C. humireducens, particularly preferably from C. humireducens.

“Overexpression” in accordance with the invention is generally understood to mean an increase in the intracellular concentration or activity of a ribonucleic acid, a protein (polypeptide) or an enzyme, which are encoded by a corresponding DNA, in a microorganism, compared to the starting strain (parent strain) or wild-type strain. A starting strain (parent strain) means the strain on which the measure leading to overexpression has been carried out.

The increase in the concentration or activity can be achieved, for example, by increasing the copy number of the corresponding coding polynucleotides, chromosomally or extrachromosomally, by at least one copy.

A widespread method for increasing the copy number consists of incorporating the corresponding coding polynucleotide into a vector, preferably a plasmid, which is replicated from a microorganism, particularly a coryneform bacteria. Furthermore, transposons, insertion elements (IS elements) or phages can be used as vectors. An abundance of suitable vectors is described in the prior art.

Another widespread method for achieving overexpression is the method of chromosomal gene amplification. In this method, at least one additional copy of the polynucleotide of interest is inserted into the chromosome of a coryneform bacterium. Such amplification methods are described for example in WO 03/014330 or WO 03/040373.

A further method for achieving overexpression consists of linking the corresponding gene or allele in a functional manner (operably linked) to a promoter or an expression cassette. Suitable promoters for Corynebacterium glutamicum are described, for example, in FIG. 1 of the review article of Patek et al. (Journal of Biotechnology 104(1-3), 311-323 (2003)) and in comprehensive reviews such as the “Handbook of Corynebacterium glutamicum” (Eds.: Lothar Eggeling and Michael Bott, CRC Press, Boca Raton, US (2005)) or the book “Corynebacteria, Genomics and Molecular Biology” (Ed.: Andreas Burkovski, Caister Academic Press, Norfolk, UK (2008)). In the same way, variants of the dapA promoter, the promoter A25 for example, described in Vasicova et al (Journal of Bacteriology 181, 6188-6191 (1999)), may be used. Furthermore, the gap promoter of Corynebacterium glutamicum (EP 06007373) may be used. Finally, the well known promoters T3, T7, SP6, M13, lac, tac and trc, described by Amann et al. (Gene 69(2), 301-315 (1988)) and Amann and Brosius (Gene 40(2-3), 183-190 (1985)), may be used. Such a promoter can be inserted, for example, upstream of the relevant gene, typically at a distance of about 1-500 nucleobases from the start codon.

The measures of overexpression increase the activity or concentration of the corresponding polypeptide preferably by at least 10%, 25%, 50%, 75%, 100%, 150%, 200%, 300%, 400% or 500%, preferably at most by 1000% or 2000%, based on the activity or concentration of said polypeptide in the strain prior to the measure resulting in overexpression.

The concentration of a protein may be determined via 1- and 2-dimensional protein gel fractionation and subsequent optical identification of the protein concentration by appropriate evaluation software in the gel. A customary method of preparing protein gels for coryneform bacteria and of identifying said proteins is the procedure described by Hermann et al. (Electrophoresis, 22:1712-23 (2001)). The protein concentration may likewise be determined by Western blot hybridization using an antibody specific for the protein to be detected (Sambrook et al., Molecular Cloning: a laboratory manual, 2nd Ed. 2nd Ed. Cold Spring Harbor Laboratory Press, Cold Spring Harbor, N.Y., 1989) and subsequent optical evaluation using corresponding software for concentration determination (Lohaus and Meyer (1998) Biospektrum 5:32-39; Lottspeich, Angewandte Chemie 38: 2630-2647 (1999)). The activity may be determined by means of a suitable enzyme assay.

“Attenuation” in accordance with the invention refers to a decrease in the intracellular concentration or activity of a ribonucleic acid, a protein (polypeptide) or an enzyme, which are encoded by a corresponding DNA, in a microorganism, compared to the starting strain (parent strain) or wild-type strain. The starting strain (parent strain) refers to the strain on which the measure for the attenuation was carried out.

The attenuation can be achieved by reducing the expression of a polypeptide, for example, by using a weak promoter or by using an allele coding for a polypeptide having a lower activity and optionally these measures may be combined. The attenuation can also be achieved by completely preventing the expression of the polypeptide, for example, by deactivating the coding gene.

The measure of attenuation decreases the activity or concentration of the corresponding polypeptide preferably by at least 10%, 25%, 50% or 75%, at most 100%, based on the activity or concentration of said polypeptide in the strain prior to the measure resulting in attenuation. In a preferred embodiment, the attenuation consists of completely deactivating the expression of the relevant polypeptide.

Feedback-resistant enzymes in connection with amino acid production is generally understood to mean enzymes which, compared to the wild form, have a lower sensitivity to inhibition by the L-amino acids and/or analogues thereof produced.

In particular, feedback-resistant aspartate kinases (LysC^{FBR}) mean aspartate kinases which, by comparison with the wild form, show less sensitivity to inhibition by mixtures of lysine and threonine or mixtures of AEC (aminoethylcysteine) and threonine or lysine alone or AEC alone. For lysine production, corresponding strains are preferably used which comprise such feedback-resistant or desensitised aspartate kinases.

For example, the following feedback-resistant aspartate kinases from C. glutamicum are known from the literature: A279T, A279V, S301F, S301Y, T308I, T311I, R320G, G345D, S381F. With respect to feedback-resistant aspartate kinases from C. glutamicum, reference is also made to the following publications: JP1993184366-A, JP1994062866-A, JP1994261766-A, JP1997070291-A, JP1997322774-A, JP1998165180-A, JP1998215883-A, U.S. Pat. No. 5,688,671-A, EP0387527, WO00/63388, U.S. Pat. No. 3,732,144, JP6261766, Jetten et al. (1995; Applied Microbiology Biotechnology 43: 76-82). Feedback-resistant aspartate kinases from C. glutamicum are deposited in the NCBI GenBank under the following accession numbers: E05108, E06825, E06826, E06827, E08177, E08178, E08179, E08180, E08181, E08182, E12770, E14514, E16352, E16745, E16746, I74588, I74589, I74590, I74591, I74592, I74593, I74594, I74595, I74596, I74597, X57226, L16848, L27125.

The following feedback-resistant aspartate kinases from C. humireducens according to the invention are preferably used: D274Y, A279E, S301Y, T308I, T311I, G359D.

For threonine production, preference is likewise given to using strains comprising a corresponding feedback-resistant homoserine dehydrogenase (Hom^{FBR}).

For isoleucine production and valine production, preference is likewise given to using strains comprising a corresponding feedback-resistant acetolactate synthase.

For leucine production, preference is likewise given to using strains comprising a corresponding feedback-resistant isopropylmalate synthase (LeuA^{FBR}).

For proline production, preference is likewise given to using strains comprising a corresponding feedback-resistant glutamate-5-kinase (ProB^{FBR}).

For arginine production, preference is likewise given to using strains comprising a corresponding feedback-resistant ornithine carbamoyltransferase (ArgF^{FBR}).

For serine production, preference is likewise given to using strains comprising a corresponding feedback-resistant D-3-phosphoglycerate dehydrogenase (SerA^{FBR}).

For methionine production, preference is likewise given to using strains comprising a corresponding feedback-resistant D-3-phosphoglycerate dehydrogenase (SerA^{FBR}) and/or feedback-resistant pyruvate carboxylases (Pyc^{FBR}).

For tryptophan production, preference is likewise given to using strains comprising a corresponding feedback-resistant phospho-2-dehydro-3-deoxyheptonate aldolase (AroG^{FBR} or AroH^{FBR}).

With regard to further more preferable properties of the L-amino acid-overproducing C. humireducens strain to be used in accordance with the invention, reference is made to the publication of Wu et al. (2011) cited above and the other publications mentioned above.

Microorganisms according to the invention, particularly bacteria of the genus Corynebacterium, may be cultured continuously—as described for example in WO 05/021772—or discontinuously in a batch process (batch cultivation or batch method) or in a fed batch or repeated fed batch process for the purpose of producing L-lysine. A general review of known cultivation methods is available in the textbook by Chmiel (Bioprozesstechnik 1. Einführung in die Bioverfahrenstechnik [Bioprocess Technology 1. Introduction to Bioprocess Technology] (Gustav Fischer Verlag, Stuttgart, 1991)) or in the textbook by Storhas (Bioreaktoren und periphere Einrichtungen [Bioreactors and Peripheral Devices] (Vieweg Verlag, Braunschweig/Wiesbaden, 1994)).

The culture medium or fermentation medium to be used has to satisfy the demands of the particular strains in a suitable manner. Descriptions of culture media of different microorganisms are present in the handbook “Manual of Methods for General Bacteriology”, of the American Society for Bacteriology (Washington D. C., USA, 1981). The terms culture medium and fermentation medium or medium are mutually interchangeable.

The carbon sources used may be sugars and carbohydrates such as glucose, sucrose, lactose, fructose, maltose, molasses, sucrose-containing solutions from sugarbeet or sugarcane production, starch, starch hydrolysate and cellulose, oils and fats such as soybean oil, sunflower oil, groundnut oil and coconut fat, fatty acids such as palmitic acid, stearic acid and linoleic acid, alcohols such as glycerol, methanol and ethanol and organic acids such as acetic acid or lactic acid.

It is possible to use, as nitrogen source, organic nitrogen-containing compounds such as peptones, yeast extract, meat extract, malt extract, corn steep liquor, soybean flour and urea, or inorganic compounds such as ammonium sulphate, ammonium chloride, ammonium phosphate, ammonium carbonate and ammonium nitrate. The nitrogen sources may be used individually or as a mixture.

The phosphorus sources used may be phosphoric acid, potassium dihydrogen phosphate or dipotassium hydrogen phosphate or the corresponding sodium-containing salts.

The culture medium must additionally contain salts, for example in the form of chlorides or sulphates of metals such as sodium, potassium, magnesium, calcium and iron, for example magnesium sulphate or iron sulphate, which are needed for growth. Finally, essential growth factors such as amino acids, for example homoserine, and vitamins, for example thiamine, biotin or pantothenic acid, may be used in addition to the substances mentioned above.

The feedstocks mentioned may be added to the culture in the form of a single mixture or may be fed in during the cultivation in a suitable manner.

The pH of the culture can be controlled by employing basic compounds such as sodium hydroxide, potassium hydroxide, ammonia or aqueous ammonia, or acidic compounds such as phosphoric acid or sulphuric acid in a suitable manner. The pH is generally adjusted to a value of 6.0 to 9.0, preferably 6.5 to 8. To control the evolution of foam, it is possible to use antifoams, for example fatty acid polyglycol esters. To maintain the stability of plasmids, it is possible to add to the medium suitable selective substances such as, for example, antibiotics. In order to maintain aerobic conditions, oxygen or oxygenous gas mixtures, for example air, are introduced into the culture. The use of liquids enriched with hydrogen peroxide is likewise possible. If appropriate, the fermentation is conducted at elevated pressure, for example at a pressure of 0.03 to 0.2 MPa. The temperature of the culture is normally 20° C. to 45° C. and preferably 25° C. to 40° C. In batch processes, the cultivation is continued until a maximum of the desired L-amino acid has formed. This aim is normally achieved within 10 hours to 160 hours. In continuous processes, longer cultivation times are possible. The activity of the bacteria results in a concentration (accumulation) of the L-amino acid in the fermentation medium and/or in the bacterial cells.

Examples of suitable fermentation media are found, inter alia, in the patent specifications 5,770,409, U.S. Pat. No. 5,840,551 and U.S. Pat. No. 5,990,350 or U.S. Pat. No. 5,275,940.

Analysis of L-amino acids to determine the concentration at one or more time(s) during the fermentation can take place by separating the L-amino acids by means of ion exchange chromatography, preferably cation exchange chromatography, with subsequent post-column derivatization using ninhydrin, as described in Spackman et al. (Analytical Chemistry 30: 1190-1206 (1958)). It is also possible to employ ortho-phthalaldehyde rather than ninhydrin for post-column derivatization. An overview article on ion exchange chromatography can be found in Pickering (LC.GC (Magazine of Chromatographic Science) 7(6), 484-487 (1989)).

It is likewise possible to carry out a pre-column derivatization, for example using ortho-phthalaldehyde or phenyl isothiocyanate, and to fractionate the resulting amino acid derivates by reversed-phase chromatography (RP), preferably in the form of high-performance liquid chromatography (HPLC). A method of this type is described, for example, in Lindroth et al. (Analytical Chemistry 51: 1167-1174 (1979)).

Detection is carried out photometrically (absorption, fluorescence).

A review regarding amino acid analysis can be found inter alia in the textbook “Bioanalytik” from Lottspeich and Zorbas (Spektrum Akademischer Verlag, Heidelberg, Germany 1998).

Accordingly, the invention relates also to a method for producing an L-amino acid, characterized in that the following steps are carried out:
a) fermentation of the microorganisms according to the invention, particularly coryneform bacteria, preferably of the genus Corynebacterium, particularly preferably of the species Corynebacterium glutamicum or Corynebacterium humireducens, in a suitable nutrient medium, and
b) accumulation of the L-amino acids in the nutrient medium and/or in the cells of the bacteria mentioned.

A product containing L-amino acid is then provided or produced or recovered in liquid or solid form.

The fermentation measures result in a fermentation broth which comprises the relevant L-amino acid.

A fermentation broth means a fermentation medium or nutrient medium in which a microorganism has been cultivated for a certain time and at a certain temperature. The fermentation medium or the media used during the fermentation comprises/comprise all of the substances or components which ensure propagation of the microorganism and formation of the desired L-amino acid.

When the fermentation is complete, the resulting fermentation broth accordingly comprises
a) the biomass (cell mass) of the microorganism, said biomass having been produced due to propagation of the cells of said microorganism,
b) the L-amino acid formed during the fermentation,
c) the organic by-products formed during the fermentation, and
d) the constituents of the fermentation medium employed or of the starting materials, such as, for example, vitamins such as biotin or salts such as magnesium sulphate, which have not been consumed in the fermentation.

The organic by-products include substances which are produced by the microorganisms employed in the fermentation in addition to the desired L-amino acid and are optionally secreted. These also include sugars such as, for example, trehalose.

The fermentation broth is removed from the culture vessel or fermentation tank, collected where appropriate, and used for providing an L-amino acid-containing product, in liquid or solid form. The expression “recovering the L-amino acid-containing product” is also used for this. In the simplest case, the L-amino acid-containing fermentation broth itself constitutes the recovered product.

One or more of the measures selected from the group consisting of
a) partial (>0% to <80%) to complete (100%) or virtually complete (≧80%, ≧90%, ≧95%, ≧96%, ≧97%, ≧98%, ≧99%) removal of the water,
b) partial (>0% to <80%) to complete (100%) or virtually complete (≧80%, ≧90%, ≧95%, ≧96%, ≧97%, ≧98%, ≧99%) removal of the biomass, the latter being optionally inactivated before removal,
c) partial (>0% to <80%) to complete (100%) or virtually complete (≧80%, ≧90%, ≧95%, ≧96%, ≧97%, ≧98%, ≧99%, ≧99.3%, ≧99.7%) removal of the organic by—products formed during fermentation, and
d) partial (>0%) to complete (100%) or virtually complete (≧80%, ≧90%, ≧95%, ≧96%, ≧97%, ≧98%, ≧99%, ≧99.3%, ≧99.7%) removal of the constituents of the fermentation medium employed or of the starting materials, which have not been consumed in the fermentation,
   from the fermentation broth achieves concentration or purification of the L-amino acid. Products having a desired content of L-amino acid are isolated in this way.

The partial (>0% to <80%) to complete (100%) or virtually complete (≧80% to <100%) removal of the water (measure a)) is also referred to as drying.

Complete or virtually complete removal of the water, of the biomass, of the organic by-products and of the unconsumed constituents of the fermentation medium employed results in pure (≧80% by weight, ≧90% by weight) or high-purity (≧95% by weight, ≧97% by weight, ≧99% by weight) product forms of the L-amino acid. An abundance of technical instructions for measures a), b), c) and d) are available in the prior art.

### WORKING EXAMPLES

### Example 1

### Alanine and Valine Production by C. Humireducens

To assess alanine and valine production, the strain C. humireducens (DSM 45392) was cultured in a shaking flask batch. For this purpose, the C. humireducens strain was incubated in 10 ml of BHI liquid medium (Brain Heart Infusion; Merck) (37 g/l H₂O) at 37° C. at 200 rpm for 24 h as preculture. 10 ml of shaking flask medium were then inoculated to an OD₆₆₀ of 0.2 and cultured at 37° C. at 200 rpm for 48 h. To prepare said medium, 20 g of ammonium sulphate, 0.4 g of MgSO₄*7H₂O, 0.6 g of KH₂PO₄ and 10 g of yeast extract were dissolved in 750 ml of H₂O. The pH of the solution was adjusted to 7.8 with 20% NH₄OH and the solution was then autoclaved. 4 ml of a vitamin solution (pH 7 with NH₄OH), consisting of 0.25 g/l of thiamine, 50 mg/l of cyanocobalamin, 25 mg/l of biotin and 1.25 g/l of pyridoxine, were then added. In addition, 140 ml of a sterile-filtered 50% glucose solution and 50 g of dry autoclaved CaCO₃ were added and the medium subsequently made up to one litre. After culturing, the supernatant of four parallel cultures was in each case analysed by HPLC to determine the alanine, glycine and valine content with a detection limit of ≧0.01 g/l.

The strain C. humireducens after culturing for 48 h in shaking flask medium at 37° C., 200 rpm at a shaking flask scale produces around 0.81 g/l of alanine (net yield: 0.011 gₐₗₐₙᵢₙₑ/g_{glucose}) and 1.6 g/l of valine (net yield: 0.022 gᵥₐₗᵢₙₑ/g_{glucose}) (Tab. 1). Glycine was only produced in small amounts as by-product.

TABLE 1
Analytical data from a shaking flask experiment with the strain
C. humireducens. The values measured after culturing with cells
and with the blank medium are shown.

| | Alanine | Valine |
|---|---|---|
| | (g/l) | (g/l) |
| C. humireducens | 1.27 | 1.9 |
| Blank medium without cells | 0.46 | 0.3 |

### Example 3

### Glutamate Performance Assay

For the L-glutamate performance assay, the strain C. humireducens (DSM 45392) was cultured in a shaking flask batch. For this purpose, the C. humireducens strain was incubated in 10 ml of BHI liquid medium (Brain Heart Infusion; Merck) (37 g/l H₂O) at 37° C. at 200 rpm for 24 h as preculture. 10 ml of shaking flask medium were then inoculated to an OD₆₆₀ of 0.2 and cultured at 37° C. at 200 rpm for 48 h. To prepare said medium, 20 g of ammonium sulphate, 0.4 g of MgSO₄*7H₂O, 0.6 g of KH₂PO₄ and 10 g of yeast extract were dissolved in 750 ml of H₂O. The pH of the solution was adjusted to 7.8 with 20% NH₄OH and the solution was then autoclaved. 4 ml of a vitamin solution (pH 7 with NH₄OH), consisting of 0.25 g/l of thiamine, 50 mg/l of cyanocobalamin, 25 mg/l of biotin and 1.25 g/l of pyridoxine, were then added. In addition, 140 ml of a sterile-filtered 50% glucose solution and 50 g of dry autoclaved CaCO₃ were added. 5 ml of a 400 mM sterile-filtered threonine stock solution were then added and the medium was subsequently made up to one litre.

After culturing, the supernatant of four parallel cultures was in each case analysed by HPLC to determine the glutamate content with a detection limit of ≧0.01 g/l.

The strain C. humireducens after culturing for 48 h in shaking flask medium at 37° C., 200 rpm at a shaking flask scale produced 1.8 (+/−0.6) g/l of L-glutamate. The initial concentration of L-glutamate in the medium was 0.78 (+/−0.1) g/l. Glycine was only produced in small amounts as by-product.

1-15. (canceled)

16. A glycine cleavage system comprising one or more of the enzymes GcvP, GcvT and GcvH, wherein:
a) GcvP comprises a sequence at least 80% identical to the sequence of SEQ ID NO:40;
b) GcvT comprises a sequence at least 80% identical to the sequence of SEQ ID NO:42; and
c) GcvH comprises a sequence at least 80% identical to the sequence of SEQ ID NO:38.

17. The glycine cleavage system of claim 16, wherein said system comprises at least two of said enzymes.

18. The glycine cleavage system of claim 16, wherein said system comprises all three of said enzymes.

19. The glycine cleavage of claim 16, wherein:
a) GcvP comprises a sequence at least 95% identical to the sequence of SEQ ID NO:40;
b) GcvT comprises a sequence at least 95% identical to the sequence of SEQ ID NO:42; and
c) GcvH comprises a sequence at least 95% identical to the sequence of SEQ ID NO:38.

20. The glycine cleavage system of claim 19, wherein said system comprises all three of said enzymes.

21. The glycine cleavage system of claim 16, wherein said system comprises at least one further polypeptide selected from the group consisting of:
a) a LipA enzyme having a sequence at least 80% identical to the sequence of SEQ ID NO:48;
b) a LipB enzyme having a sequence at least 80% identical to the sequence of SEQ ID NO:50;
c) a Lpd enzyme having a sequence at least 80% identical to the sequence of SEQ ID NO:52;
d) a LplA enzyme having a sequence at least 80% identical to the sequence of SEQ ID NO:94;
e) a GcvL enzyme having a sequence at least 80% identical to the sequence of SEQ ID NO:96.

22. The glycine cleavage system of claim 21, wherein said system comprises all three of said enzymes and wherein:
a) GcvP comprises a sequence at least 95% identical to the sequence of SEQ ID NO:40;
b) GcvT comprises a sequence at least 95% identical to the sequence of SEQ ID NO:42; and
c) GcvH comprises a sequence at least 95% identical to the sequence of SEQ ID NO:38.

23. The glycine cleavage system of claim 16, wherein said system comprises:
a) GcvP comprising a sequence at least 96% identical to the sequence of SEQ ID NO:40;
b) GcvT comprising a sequence at least 96% identical to the sequence of SEQ ID NO:42;
c) GcvH comprising a sequence at least 96% identical to the sequence of SEQ ID NO:38;
d) LipA comprising a sequence at least 96% identical to the sequence of SEQ ID NO:48;
e) LipB comprising a sequence at least 96% identical to the sequence of SEQ ID NO:50;
f) Lipd comprising a sequence at least 96% identical to the sequence of SEQ ID NO:52;
g) LilA comprising a sequence at least 96% identical to the sequence of SEQ ID NO: 94;
h) GcvL comprising a sequence at least 96% identical to the sequence of SEQ ID NO:96.

24. A recombinant microorganism, comprising the glycine cleavage system of claim 16.

25. The recombinant microorganism of claim 24, wherein one or more polynucleotides encoding the enzymes in the glycine cleavage system are overexpressed.

26. The recombinant microorganism of claim 24, wherein said microorganism overproduces L-methionine and comprises one or more of the of the following features:
a) an attenuated polynucleotide (mcbR), which codes for a DNA binding domain having a sequence at least 95% identical to the sequence of SEQ ID NO:2;
b) an attenuated polynucleotide (thrB gene), which codes for a homoserine kinase having a sequence at least 95%, identical to the sequence of SEQ ID NO:4;
c) an attenuated polynucleotide (pgi), which codes for a glucose-6-phosphate isomerase having a sequence at least 95%, identical to the sequence of SEQ ID NO:6;
d) an attenuated polynucleotide (pck), which codes for a phosphoenol-pyruvate carboxykinase having a sequence at least 95%, identical to the sequence of SEQ ID NO:8;
e) an attenuated polynucleotide (metQ), which codes for a D-methionine-binding lipoprotein having a sequence at least 95%, identical to the sequence of SEQ ID NO:10;
an attenuated polynucleotide (metP), which codes for a methionine transporter having a sequence at least 95%, identical to the sequence of SEQ ID NO:12;
g) an attenuated polynucleotide (metN), which codes for an ATP-dependent methionine transporter having a sequence at least 95% identical to the sequence of SEQ ID NO:14;
h) an attenuated polynucleotide (metK), which codes for an S-adenosyl-methionine synthase having a sequence at least 95% identical to the sequence of SEQ ID NO:16;
i) an attenuated polynucleotide (metI), which codes for a methionine import system permease having a sequence at least 95% identical to the sequence of SEQ ID NO:18;
j) an attenuated polynucleotide (dapA), which codes for a 4-hydroxy-tetrahydrodipicolinate synthase having a sequence at least 95% identical to the sequence of SEQ ID NO:20;
k) an overexpressed polynucleotide (CBS), which codes for a cysteine synthase having a sequence at least 95% identical to the sequence of SEQ ID NO:22;
l) an attenuated polynucleotide, which codes for a cg3031 homologue having a sequence at least 95% identical to the sequence of SEQ ID NO:24;
m) an overexpressed polynucleotide (aecD), which codes for a cystathionine beta-lyase having a sequence at least 95% identical to the sequence of SEQ ID NO:26;
n) an overexpressed polynucleotide (asd), which codes for an aspartate semialdehyde dehydrogenase having a sequence at least 95% identical to the sequence of SEQ ID NO:28;
o) an overexpressed polynucleotide (metH), which codes for a 5-methyltetra-hydrofolate homocysteine methyltransferase (MetH, EC 2.1.1.13);
P) an overexpressed polynucleotide (brnE), which codes for the smaller subunit of a transporter for branched-chain amino acids (BrnE) and having a sequence identity at least 95%, identical to the sequence of SEQ ID NO:30;
q) an overexpressed polynucleotide (brnF), which codes for the larger subunit of a transporter for branched-chain amino acids (BrnF) having a sequence at least 95% identical to the sequence of SEQ ID NO:32;
r) an overexpressed polynucleotide (cysE), which codes for a serine acetyl-transferase (CysE) having a sequence at least 95% identical to the sequence of SEQ ID NO:34;
s) an overexpressed polynucleotide (cysK), which codes for a cysteine synthase (CysK) having a sequence at least 95% identical to the sequence of SEQ ID NO:36;
t) an overexpressed polynucleotide (glyA), which codes for a serine hydroxymethyltransferase (GlyA) having a sequence at least 95% identical to the sequence of SEQ ID NO:44;
u) an overexpressed polynucleotide (horn), which codes for an optionally feedback-resistant homoserine dehydrogenase (Horn) having a sequence at least 95% identical to the sequence of SEQ ID NO:46;
v) an overexpressed polynucleotide (lysC), which codes for an optionally feedback-resistant aspartate kinase (LysC) having a sequence at least 95%, identical to the sequence of SEQ ID NO:54;
w) an overexpressed polynucleotide (metB), which codes for a cystathionine gamma-synthase (MetB) having a sequence at least 95% identical to the sequence of SEQ ID NO:56;
x) an overexpressed polynucleotide (metF), which codes for a 5,10-methylene-tetrahydrofolate reductase (MetF) having a sequence at least 95% identical to the sequence of SEQ ID NO:58;
y) an overexpressed polynucleotide (metX), which codes for a homoserine O-acetyltransferase (MetX) having a sequence at least 95% identical to the sequence of SEQ ID NO:60;
z) an overexpressed polynucleotide (metY), which codes for an O-acetylhomoserine lyase (MetY) having a sequence at least 95% identical to the sequence of SEQ ID NO:62;
aa) an overexpressed polynucleotide (pyc), which codes for a pyruvate carboxylase (Pyc) having a sequence at least 95% identical to the sequence of SEQ ID NO:64;
bb) an overexpressed polynucleotide (serA), which codes for an optionally feedback-resistant D-3-phosphoglycerate dehydrogenase (SerA) having a sequence at least 95% identical to the sequence of SEQ ID NO:66;
cc) an overexpressed polynucleotide (serB), which codes for a phosphoserine phosphatase (SerB) having a sequence at least 95% identical to the sequence of SEQ ID NO:68;
dd) an overexpressed polynucleotide (serC), which codes for a phosphoserine aminotransferase (SerC) having a sequence at least 95% identical to the sequence according of SEQ ID NO:70;
ee) an overexpressed polynucleotide (ald), which codes for an alanine dehydrogenase (Ald) having a sequence at least 95% identical to the sequence of SEQ ID NO:72;
ff) an overexpressed polynucleotide (cysD), which codes for the subunit of a sulphate adenylyltransferase (CysD) having a sequence at least 95% identical to the sequence of SEQ ID NO:74;
gg) an overexpressed polynucleotide (cysH), which codes for an adenosine phosphosulphate reductase (CysH) having a sequence at least 95% identical to the sequence of SEQ ID NO:76;
hh) an overexpressed polynucleotide (cysI), which codes for a sulphite reductase (CysI) having a sequence at least 95% identical to the sequence of SEQ ID NO:78;
ii) an overexpressed polynucleotide (cysJ), which codes for (CysJ) having a sequence at least 95% identical to the sequence of SEQ ID NO:80;
jj) an overexpressed polynucleotide (cysN), which codes for the subunit of a sulphate adenylyltransferase (CysN) having a sequence at least 95% identical to the sequence of SEQ ID NO:82;
kk) an overexpressed polynucleotide (cysY), which codes for a cystathionine beta-synthase (CysY) having a sequence at least 95% identical to the sequence of SEQ ID NO:84;
ll) an overexpressed polynucleotide (cysZ), which codes for a putative sulphate transporter (CysZ) having a sequence at least 95% identical to the sequence of SEQ ID NO:86;
mm) an overexpressed polynucleotide (metE), which codes for a 5-methyltetrahydropteroyltriglutamate-homocysteine methyltransferase (MetE) having a sequence at least 95% identical to the sequence of SEQ ID NO:88;
nn) an overexpressed polynucleotide (ptH1), which codes for a peptidyl-tRNA hydrolase 1 (PtH1) having a sequence at least 95% identical to the sequence of SEQ ID NO:90;
oo) an overexpressed polynucleotide (ptH2), which codes for a peptidyl-tRNA hydrolase 2 (PtH2) having a sequence at least 95% identical to the sequence of SEQ ID NO:92.

27. The recombinant microorganism of claim 24, wherein said microorganism is a Corynebacterium.

28. A method for overproducing an L-amino acid using a recombinant microorganism comprising the glycine cleavage system of claim 16.

29. The method of claim 28, wherein the recombinant microorganism overproduces an L-amino acid selected from the group consisting of: L-alanine, L-valine, L-amino acids of the glutamate family, L-glutamate, L-glutamine, L-proline and L-arginine, L-aspartate, L-asparagine, L-methionine, L-lysine, L-isoleucine and L-threonine.

30. The method of claim 29, wherein said L-amino acid is L-methionine.

31. The method of claim 29, wherein only low amounts of glycine occur as a by-product.
